(19) United States
(12) Patent Application Publication
(10) Pub. No.: US 2016/0194279 A1
(43) Pub. Date: Jul. 7, 2016

(54) ONE POT PROCESS FOR THE CONVERSION OF AROYL CHLORIDES TO ACYL THIOUREAS

(71) Applicant: COUNCIL OF SCIENTIFIC & INDUSTRIAL RESEARCH, New Delhi (IN)

(72) Inventors: VENKATA RAMANA CHEPURI, PUNE (IN); SENTHILKUMAR BEERAN, PUNE (IN)

(21) Appl. No.: 14/650,778

(22) PCT Filed: Dec. 9, 2013
(86) PCT No.: PCT/IN2013/000758

§ 371 Date: Jun. 9, 2015

(30) Foreign Application Priority Data
Dec. 9, 2012 (IN) 3482/DEL/2012

(51) Int. Cl. C07C 335/26

(52) U.S. Cl. CPC C07C 335/26

(57) ABSTRACT

The present invention disclose an improved one pot process for synthesis of acyl thioureas of formula (I), with yield greater than 80%, from aroyl chlorides of formula (I) wherein, R′ is an aryl or a heteroarylene group substituted with one or more groups selected from hydrogen, alkyl, alkylene, alkynyl, alkoxy, alkenyloxy, halo, hydroxyl, nitro, amino, carboxyl, ester, halogenated hydrocarbon or an aryl or heteroaryl; R″ and R′″ are selected independently from hydrogen, alkyl, alkylene, alkynyl, alkoxy, alkenyloxy, halo, hydroxyl, nitro, amino or halogenated hydrocarbon.

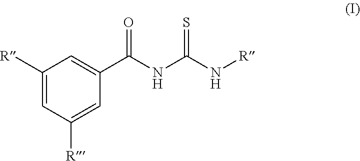

### TECHNICAL FIELD OF THE INVENTION

The present invention relates to one pot process for the conversion of aroyl chlorides to acyl thioureas with step economy. Particularly, the invention relates to an improved one pot process for synthesis of library of acyl thioureas from aroyl chlorides, wherein the yield of acylthioureas is >80%.

### BACKGROUND AND PRIOR ART OF THE INVENTION

Thioureas and their derivatives have found extensive applications in the field of medicine, agriculture and analytical chemistry, and the wide spectrum of biological activities associated with such compounds has also been reviewed in the past. These acylthioureas have been identified as potential antagonists for the PIP3. There are several methods reported for the synthesis of acylthioureas. The reported methods generally involve a two stage synthesis for the preparation of acylthioureas. A one-pot procedure employing acetone as a solvent has been documented earlier. However, the reactions in acetone in general provide several other side products. An article titled “Synthesis and biological activity of N-aroyl-N′-substituted thiourea derivatives” by You-Ming Zhang et al in Synthetic communications 31(20), 3099-3105 (2001) reports preparation of a series of N-aroyl-N′-substituted thiourea derivatives in good to excellent yields under the condition of solid-liquid phase transfer catalysis using polyethylene glycol-400(PEG-400) as the catalyst.

Another article titled “An Efficient Synthesis of Polymethylene-bis-Aroyl Thiourea Derivatives under the Condition of Phase Transfer Catalysis” by Tai-Bao Wei et al in Synthetic communications 30(3), 493-500 (2000) reports Reaction of polymethylene diamine with aroyl chloride and ammonium thiocyanate under the condition of solid-liquid phase transfer catalysis using polyethylene glycol-400 (PEG-400) as the catalyst yielded polymethylene-bis-aroyl thiourea derivatives in good to excellent yields.

Another article titled “Synthesis and anti-oxidant activity of some N-(anilinocarbonothioyl) benzamide and heterocyclic based thiourea derivatives” by P. Venkatesh et al in International Journal of ChemTech Research (Vol. 1, No. 3, pp 733-741, July-September 2009) describes a series of novel N-(anilinocarbonothioyl) benzamide prepared by reacting benzoyl chloride with ammonium thiocyanate, followed by treating the reaction mass with substituted aromatic amine to obtain N-(anilinocarbonothioyl) benzamide compounds, as shown in the below scheme 1.

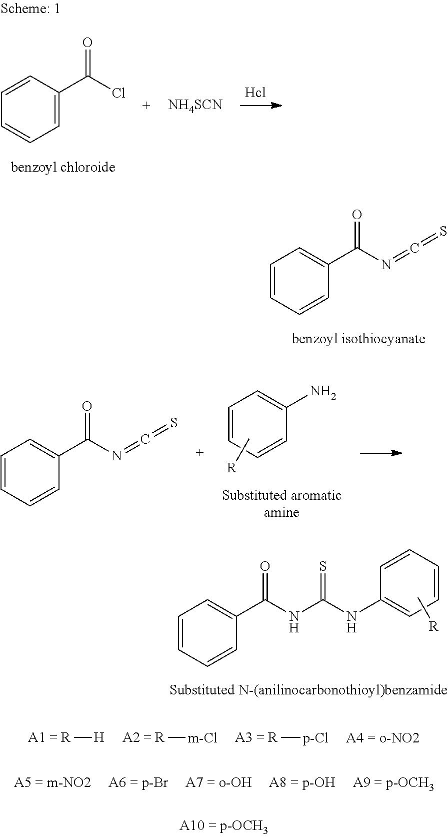

In yet another article titled “Synthesis of N-Aroyl-N′-hydroxyethyl (Hydroxyphenyl) Thiourea Derivatives under the Condition of Phase Transfer Catalysis” by Taibao Weia et al in Synthetic Communications 28(15), 2851-2859 (1998) reports reaction of aminoethanol or aminophenol with aroylchloride and ammonium thiocyanate under the condition of solid-liquid phase transfer catalysis using polyethylene glycol-600 (PEG-600) as the catalyst yielded N-aroyl-N′-hydroxyethyl (hydroxy phenyl) thiourea derivatives.

In yet another article titled “Biological Activities Studies and Phase Transfer Catalysts Promoting the One-Pot Synthesis of N-Aryl-N′-(4-Ethyloxy Benzoyl)-Thiourea Derivatives” by Jing-Han Hu in Phosphorus, Sulfur, and Silicon, 181:2691-2698, 2006, describes a one-pot facile, efficient, and high-yield method for the synthesis of N-aryl-N-(4-ethyloxybenzoyl)-thiourea under the condition of solid-liquid phase transfer catalysis using polyethylene glycol-400 (PEG-400) as the catalyst as shown in Scheme 2.

Scheme 2

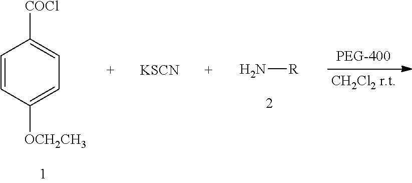

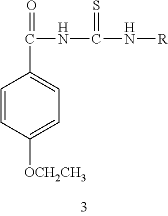

| 3 | R |
|---|---|
| 3a | 3-NO₂C₆H₄ |
| 3b | 2-MeOC₆H₄ |
| 3c | 3-MeC₆H₄ |
| 3d | —(CH₂)₂OH |
| 3e | 2-HOC₆H₄ |
| 3f | 3-HOC₆H₄ |
| 3g | 4-HOC₆H₄ |
| 3h | 4-CH₃COC₆H₄ |

In yet another article titled “Improved Procedures for the Preparation of Cycloalkyl-, Arylalkyl-, and Arylthioureas” by C. R. Rasmussen et al in Synthesis (June 1988) page 456 to 459 discloses an improved procedure for the preparation of arylthioureas consists of the reaction of benzoyl isothiocyanate with anilines in acetone to obtain N-aryl-N-benzoylthioureas as shown below in scheme 3:

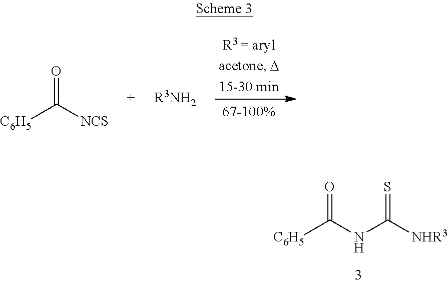

References may be made to Journal, “Synthesis and Crystal Structure of 1-(3-fluorophenyl)-3-(3,4,5-trimethoxybenzoyl)thiourea” by Aamer Saeed, Uzma Shaheen and Michael Bolte wherein thiourea was synthesized by reaction of 3,4,5-trimethoxybenzoyl isothiocyante with 3-fluoroaniline. The 3,4,5-trimethoxybenzoyl isothiocyante was produced in situ by reaction of 3,4,5-trimethoxybenzoyl chloride with ammonium thiocyanate in dry acetonitrile. The reaction is performed under reflux conditions.

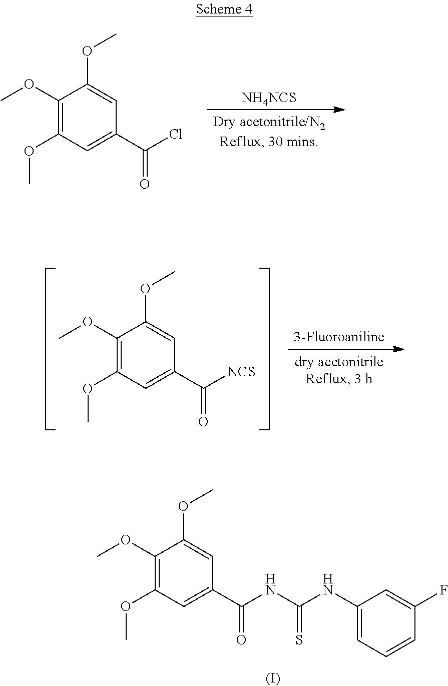

From the above, it is clear that the many of the prior arts utilizes acetone, methylene chloride and prepared thioureas under solid-liquid phase transfer catalyst, like PEG-400 as phase transfer catalyst to obtain the desired thioureas, however, the same results in poor yields and by products.

In view of the above, there is a need in the art to provide an improved one pot process for the conversion of aryl acidchlorides to acylthioureas with step economy using a suitable solvent system under mild conditions.

In accordance with the above objectives, the instant inventors have screened a variety of solvents and found that acetonitrile is the best amongst various solvents screened and the reactions proceeded smoothly and provided the requisite acylthioureas as sole products in single step.

### OBJECTIVE OF THE INVENTION

Main object of the present invention is to provide an improved one pot process for the conversion of aryl acidchlorides to acylthioureas with step economy. Another objective of the present invention is to provide an improved one pot process for synthesis of library of acyl thiourea compounds from aryl acid chlorides or aroyl chloride using a suitable solvent system under mild conditions, wherein the yield of acylthioureas is >80%.

### SUMMARY OF THE INVENTION

Accordingly, present invention provides a one pot room temperature process for preparation of acyl thioureas of formula (I) with yield greater than 80%, comprising the steps of:

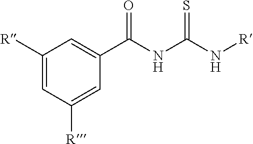

wherein, R′ is an aryl or a heteroarylene group substituted with one or more groups selected from hydrogen, alkyl, alkylene, alkynyl, alkoxy, alkenyloxy, halo, hydroxyl, nitro, amino, carboxyl, ester, halogenated hydrocarbon or an aryl or heteroaryl; R″ and R′″ are selected independently from hydrogen, alkyl, alkylene, alkynyl, alkoxy, alkenyloxy, halo, hydroxyl, nitro, amino or halogenated hydrocarbon;
i. mixing aroyl chloride of formula (II) with ammonium isothiocyanate in the ratio ranging between 1:1 to 1:3 in acetonitrile followed by stirring for period in the range of 3 to 5 minutes to obtain isothiocyanate;

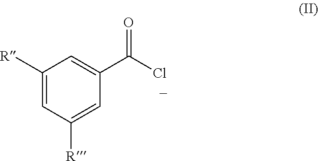

ii. adding isothiocyanate as obtained in step (i) with solution of an amine of general formula R′—NH₂ in acetonitrile in the ratio ranging between 1:0.5 to 1:1.5 followed by stirring for period in the range of 3 to 4 hr at room temperature in the range of 20 to 30° C. to obtain acyl thioureas of formula (I).

In an embodiment of the present invention, representative compounds of formula (I) comprising:
N-(2-hydroxyphenylcarbamothioyl)-3, 5-bis(trifluoromethyl)benzamide
N-(2-hydroxy-5-nitrophenylcarbamothioyl)-3,5-bis(trifluoromethyl)benzamide
N-(2-hydroxy-4-nitrophenylcarbamothioyl)-3,5-bis(trifluoromethyl)benzamide
N-(4-chloro-2-hydroxyphenylcarbamothioyl)-3, 5-bis(trifluoromethyl)benzamide
N-(5-chloro-2-hydroxyphenylcarbamothioyl)-3,5-bis(trifluoromethyl)benzamide
N-(2-hydroxy-5-methylphenylcarbamothioyl)-3, 5-bis(trifluoromethyl)benzamide
N-(2-hydroxy-4-methylphenylcarbamothioyl)-3, 5-bis(trifluoromethyl)benzamide
N-(2-hydroxy-3-nitrophenylcarbamothioyl)-3,5-bis(trifluoromethyl)benzamide
N-(2-hydroxy-5-(trifluromethyl)phenylcarbamothioyl)-3, 5-bis(trifluoromethyl)benzamide
N-(3-bromo-6-hydroxy-2,4-dimethylphenylcarbamothioyl)-3,5bis(trifluoromethyl) benzamide
N-(2-hydroxy-4,6-bis(trifluromethyl)phenylcarbamothioyl)-3,5 bis(trifluoromethyl) benzamide
N-((4-hydroxy-[1,1′-biphenyl]-3-yl)carbamothioyl)-3,5-bis(trifluoromethyl)benzamide
N,N′-thiocarbonylbis(3,5-bis(trifluoromethyl)benzamide)
N-(4-chloro-2-hydroxy-5-nitrophenylcarbamothioyl)-3,5-bis(trifluoromethyl)benzamide
N-((4-methoxy-[1,1′-biphenyl]-3-yl)carbamothioyl)-3,5-bis(trifluoromethyl)benzamide
N-(2-methoxy-5-(trifluromethyl)phenylcarbamothioyl)-3,5-bis (trifluoromethyl) benzamide
N-(2-hydroxyphenylcarbamothioyl)-3,5-dimethoxybenzamide
N-(4-chloro-2-hydroxyphenylcarbamothioyl)-3,5-dimethoxybenzamide
N-(5-chloro-2-hydroxyphenylcarbamothioyl)-3,5-dimethoxybenzamide
N-(2-hydroxy-4-methylphenylcarbamothioyl)-3,5-dimethoxybenzamide
N-((4-hydroxy-[1,1′-biphenyl]-3-yl)carbamothioyl)-3,5-dimethoxybenzamide
N-(2-hydroxy-5-(trifluromethyl)phenylcarbamothioyl)-3,5-dimethoxybenzamide
3,5-dimethoxy-N-(2-methoxy-5-(trifluoromethyl)phenylcarbamothioyl)benzamide
N-(4-iodo-2-nitrophenylcarbamothioyl)-3,5-dimethylbenzamide
3,5-dimethyl-N-(3-nitrophenylcarbamothioyl)benzamide
Methyl 2-(3-(3,5-dimethylbenzoyl)thioureido)benzoate
N-(2-hydroxynaphthalen-1-ylcarbamothioyl)-3,5-dimethylbenzamide
N-(3-chloro-6-hydroxy-2,4-dimethylphenylcarbamothioyl)-3,5-dimethylbenzamide
N-(4-fluoro-2-hydroxyphenylcarbamothioyl)-3,5-dimethylbenzamide
N-(2-hydroxy-4-nitrophenylcarbamothioyl)-3,5-dimethylbenzamide
N-(2-hydroxy-5-nitrophenylcarbamothioyl)-3,5-dimethylbenzamide
N-(2-hydroxy-4-methylphenylcarbamothioyl)-3,5-dimethylbenzamide
N-(2-hydroxy-6-nitrophenylcarbamothioyl)-3, 5-dimethylbenzamide
N-(2-hydroxy-5-methylphenylcarbamothioyl)-3,5-dimethylbenzamide
N-(5-chloro-2-hydroxyphenylcarbamothioyl)-3,5-dimethylbenzamide
N-(5-chloro-2-hydroxy-4-nitrophenylcarbamothioyl)-3,5-dimethylbenzamide
N-(4-chloro-2-hydroxyphenylcarbamothioyl)-3,5-dimethylbenzamide
N-(3-chloro-6-methoxy-2,4-dimethylphenylcarbamothioyl)-3,5-dimethylbenzamide
N-(2-hydroxy-5-(trifluoromethyl)phenylcarbamothioyl)-3,5-dimethylbenzamide
N-(2-methoxy-5-(trifluoromethyl)phenylcarbamothioyl)-3,5-dimethylbenzamide
N-(2-chloro-5-nitrophenylcarbamothioyl)-3,5-dimethylbenzamide
N-(2-hydroxy-4,6-dimethylphenylcarbamothioyl)-3,5-dimethylbenzamide
N-(2-hydroxy-3,4,6-trimethylphenylcarbamothioyl)-3,5-dimethylbenzamide
N-(6-hydroxy-2,3,4-trimethylphenylcarbamothioyl)-3,5-dimethylbenzamide
N-(1-hydroxynaphthalen-2-ylcarbamothioyl)-3,5-dimethylbenzamide
3-(3-(3,5-dimethylbenzoyl)thioureido)-4-hydroxybenzoic acid
N-(2,6-dihydroxyphenylcarbamothioyl)-3,5-dimethylbenzamide
N-(2-hydroxy-4,6-bis(trifluoromethyl)phenylcarbamothioyl)-3,5-dimethylbenzamide
N-(3-bromo-6-hydroxy-2,4-dimethylphenylcarbamothioyl)-3,5-dimethylbenzamide
N-(4-hydroxybiphenyl-3-ylcarbamothioyl)-3,5-dimethylbenzamide.
   In another embodiment of the present invention, yield of the said acyl thioureas of formula (I) is in the range of 82% to 97%.
   In yet another embodiment of the present invention, wherein aroyl chloride of formula (II) and ammonium isothiocyanate used is in the ratio ranging between 1:1 to 1:3 preferably 1:2.

### DETAILED DESCRIPTION OF THE INVENTION

The present invention provides an improved one pot process for synthesis of library of acylthiourea compounds of formula (I) from aryl acid chlorides or aroyl chloride (II) using acetonitrile as solvent wherein the yield of acylthiourea is >80%. The compound of formula (I) is represented below:

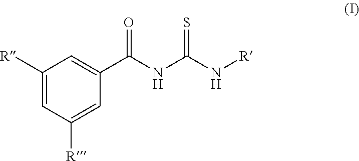

wherein, R′ is an aryl or a heteroarylene group substituted with one or more groups selected from hydrogen, alkyl, alkylene, alkynyl, alkoxy, alkenyloxy, halo, hydroxyl, nitro, amino, carboxyl, ester, halogenated hydrocarbon or an aryl or heteroaryl; R″ and R″ are selected independently from hydrogen, alkyl, alkylene, alkynyl, alkoxy, alkenyloxy, halo, hydroxyl, nitro, amino or halogenated hydrocarbon.

The preparation of library of acylthiourea compounds of formula (I) according to the invention involves in preparation of aromatic nitro compounds followed by reduction to obtain amino compounds and subsequent conversion of amino compounds into acylthiourea compounds by treatment with isothiocyanate.

Benzoyl chloride is reacted with ammonium thiocyanate in acetonitrile under stirring for 5 minutes to form white precipitation of isothiocyanate, which is subsequently reacted with amino substituted aromatic compound in acetonitrile under stirring at room temperature (20-40° C.) for 24 h. Solvent is evaporated under reduced pressure and the residue is purified by silica gel chromatography to produce thiourea coupling product.

The process scheme is given below:

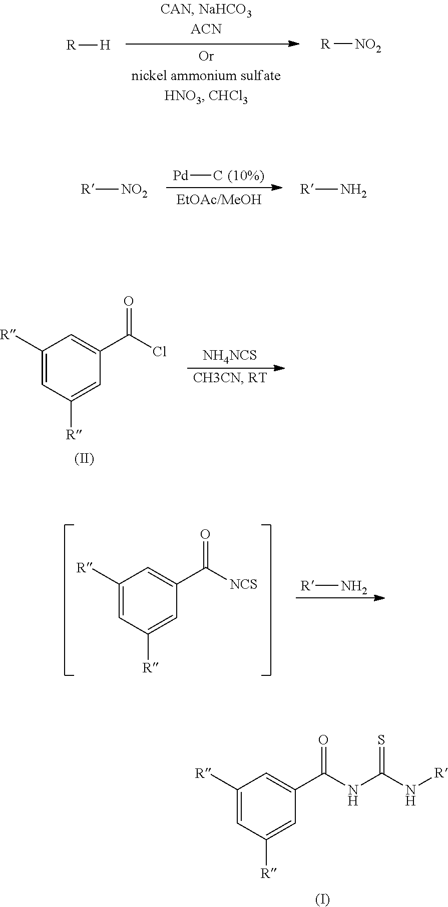

A library of compounds synthesized by the instant process and its characterization are given in the example below.

Accordingly, the invention discloses preparation of substituted aromatic amino compound, by subjecting corresponding nitro compound to reduction to obtain amino substituted compound. The reduction may be carried in presence of any metal catalysts known in the art, for example, palladium/carbon under hydrogen atmosphere (1-2 atp) for 5-6 h.

Accordingly, the substituted aromatic nitro compound as mentioned above may be prepared by treating substituted aromatic compound with CAN (Ceric ammonium nitrate) in presence of sodium bicarbonate to obtain nitro compound. Alternately, substituted aromatic nitro compound may be prepared by treating substituted aromatic compound with ammonium nickel sulfate followed by treatment with nitric acid to obtain nitro substituted compound.

### EXAMPLES

The following examples are given by way of illustration and therefore should not be construed to limit the scope of the present invention.

### Example 1

### General Procedure for Preparation of Nitro Compound

### Experimental Procedure A

To a suspension of phenol (3.5 mmol) and NaHCO₃ (14 mmol) in anhydrous MeCN (10 ml) CAN (70 mmol) was added and the resulting mixture was stirred for 30 min at 26° C. (rt). During this time, the yellow color of CAN has been disappeared. The reaction mixture was diluted with dichloromethane (25 mL) and filtered. The organic layer was washed with water (2×10 mL), dried (NaSO₄) and concentrated under reduced pressure. The resulting crude was purified by column chromatography (silica 100-200) to procure pure nitrophenol product.

### Example 2

### Experimental Procedure B

The phenol (1.0 mmol) and ammonium nickel sulfate (1.0 mmol) were dissolved in chloroform (10 ml) and treated with 69% HNO₃ (1.0 mmol) and stirred at 26° C. for 3 h. After the completion of the reaction, the reaction mixture was diluted with chloroform (10 ml), filtered. The organic layer was washed with water, dried (Na₂SO₄) and solvent evaporated under vacuum. The crude product was purified by silica gel chromatography. The process is schematically represented below.

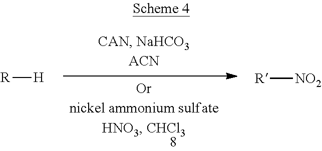

Using the above procedure many nitro compounds were prepared as per the table 1 below:

TABLE 1

| Entry | R | Product |
|---|---|---|
| 1 | 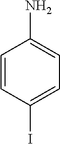 | 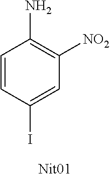 [SMILES: Nc1ccc(I)cc1[N+](=O)[O-]] |
| 2 | 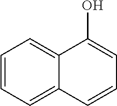 | 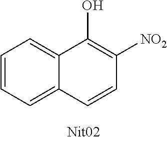 [SMILES: O=[N+]([O-])c1ccc2ccccc2c1O] |
| 3 | 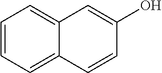 | 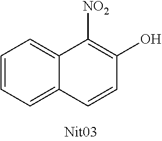 [SMILES: O=[N+]([O-])c1c(O)ccc2ccccc12] |
| 4 | 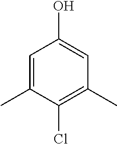 | 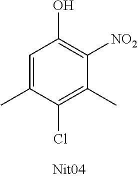 |
| 5 | 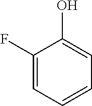 | 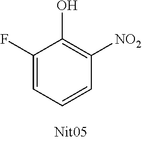 [SMILES: O=[N+]([O-])c1cccc(F)c1O] |
| 6 | 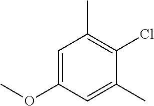 | 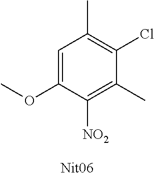 |
| 7 | 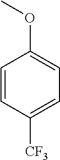 | 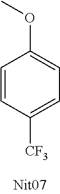 |
| 8 | 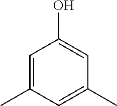 | 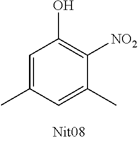 |
| 9 | 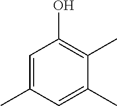 | 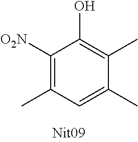 |
| 10 | 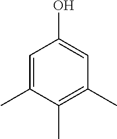 | 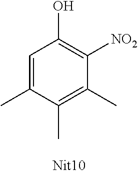 |
| 11 | 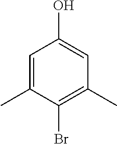 | 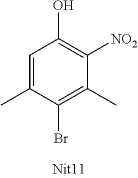 |
| 12 | 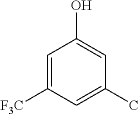 | 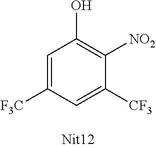 [SMILES: O=[N+]([O-])c1c(O)cc(C(F)(F)F)cc1C(F)(F)F] |

### Example 3

### General Procedure for Preparation of Amino Compound

### Experimental Procedure C

A suspension of nitro compound and Pd—C(10%) in EtOAc/MeOH was stirred under hydrogen atmosphere (1-2 atm). After completion of reaction, the reaction mixture was filtered and concentrated under reduced pressure. The resulting crude was purified by silica gel (100-200) chromatography to produce the requisite aromatic amino compounds as shown in scheme 5.

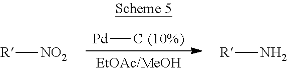

Many amino compounds prepared as per the procedure above are listed in table 2.

| Entry | R′ | Product |
|---|---|---|
| 1 | 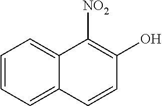 | 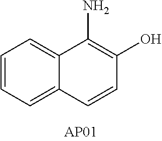 [SMILES: Nc1c(O)ccc2ccccc12] |
| 2 | 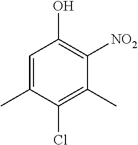 | 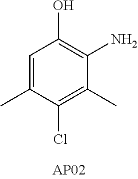 |
| 3 | 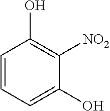 | 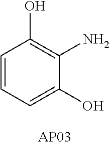 |
| 4 | 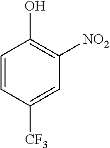 | 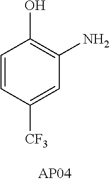 [SMILES: Nc1cc(C(F)(F)F)ccc1O] |
| 5 | 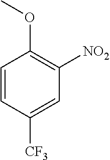 | 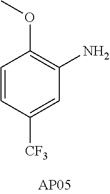 |
| 6 | 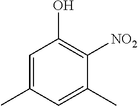 | 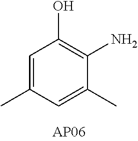 |
| 7 | 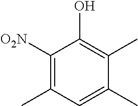 | 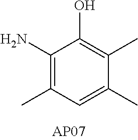 |
| 8 | 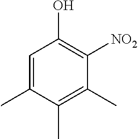 | 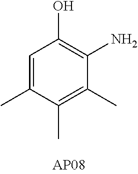 |
| 9 | 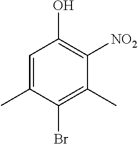 | 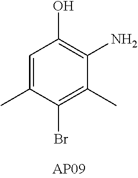 |
| 10 | 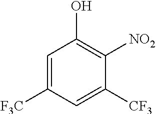 | 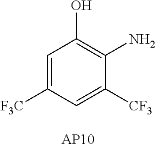 [SMILES: Nc1c(O)cc(C(F)(F)F)cc1C(F)(F)F] |

### Example 4

### General Procedure for One Pot Synthesis of Thiourea Derivatives

### Experimental Procedure D

A Solution of ammonium thiocyanate (55 mg, 0.72 mmol) in 10 mL of acetonitrile and 3,5-bis(trifluromethyl)benzoyl chloride (0.36 mmol) was added dropwise, the mixture was stirred for 5 minutes to form white precipitation of isothiocyanate, and then a solution of amine (0.36 mmol) in acetonitrile (5 mL) was added slowly. The reaction mixture was stirred at 26° C. (RT) for 3 to 4 hr. Solvent was evaporated under reduced pressure. The residue was purified by silica gel chromatography to produce thiourea coupling product. The process is outlined in scheme 6 below:

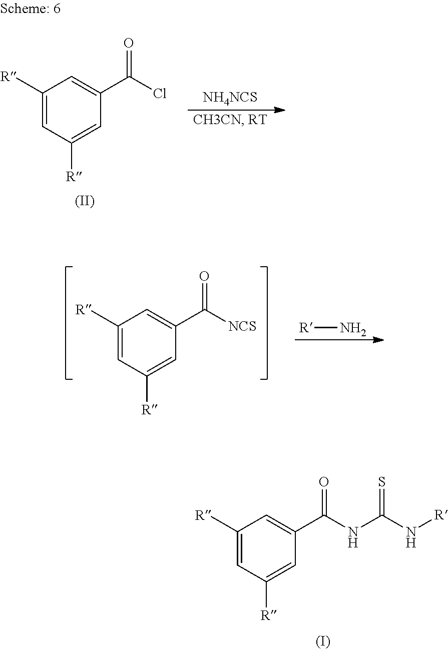

By using the above procedure library of compounds was synthesized and characterized as shown below.

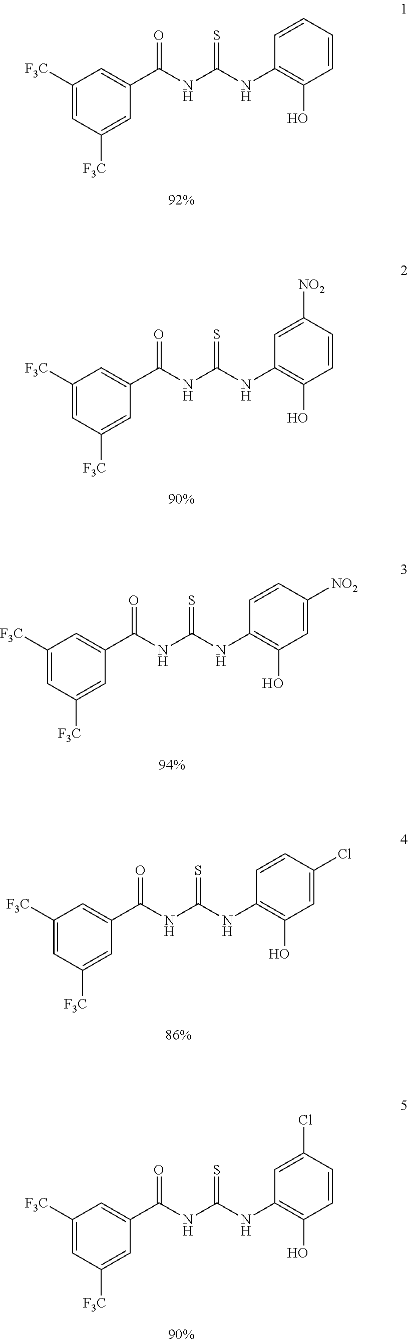 [SMILES: O=C(NC(=S)Nc1ccccc1O)c1cc(C(F)(F)F)cc(C(F)(F)F)c1 ; O=C(NC(=S)Nc1cc([N+](=O)[O-])ccc1O)c1cc(C(F)(F)F)cc(C(F)(F)F)c1 ; O=C(NC(=S)Nc1ccc([N+](=O)[O-])cc1O)c1cc(C(F)(F)F)cc(C(F)(F)F)c1 ; O=C(NC(=S)Nc1ccc(Cl)cc1O)c1cc(C(F)(F)F)cc(C(F)(F)F)c1 ; O=C(NC(=S)Nc1cc(Cl)ccc1O)c1cc(C(F)(F)F)cc(C(F)(F)F)c1]

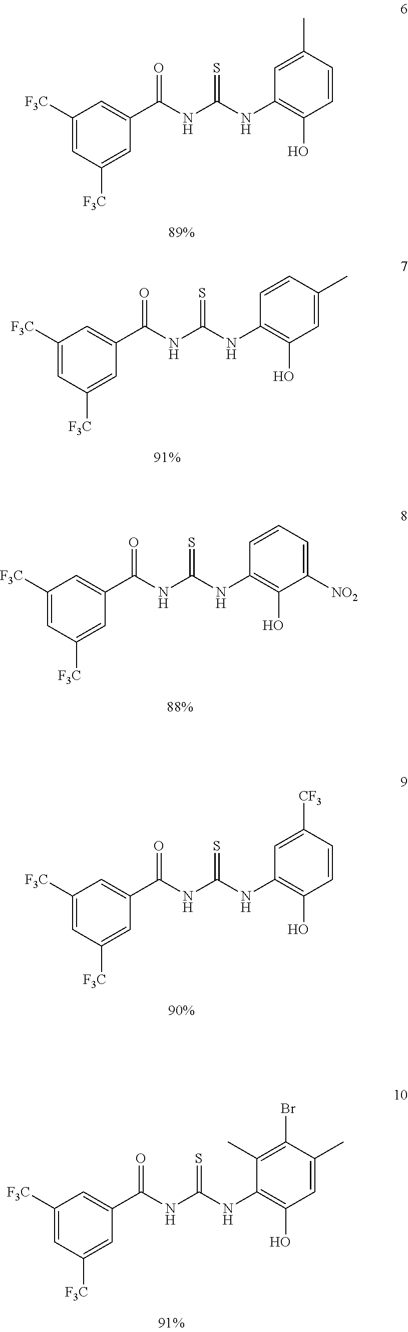 [SMILES: Cc1ccc(O)c(NC(=S)NC(=O)c2cc(C(F)(F)F)cc(C(F)(F)F)c2)c1 ; Cc1ccc(NC(=S)NC(=O)c2cc(C(F)(F)F)cc(C(F)(F)F)c2)c(O)c1 ; O=C(NC(=S)Nc1cccc([N+](=O)[O-])c1O)c1cc(C(F)(F)F)cc(C(F)(F)F)c1 ; O=C(NC(=S)Nc1cc(C(F)(F)F)ccc1O)c1cc(C(F)(F)F)cc(C(F)(F)F)c1 ; Cc1cc(O)c(NC(=S)NC(=O)c2cc(C(F)(F)F)cc(C(F)(F)F)c2)c(C)c1Br]

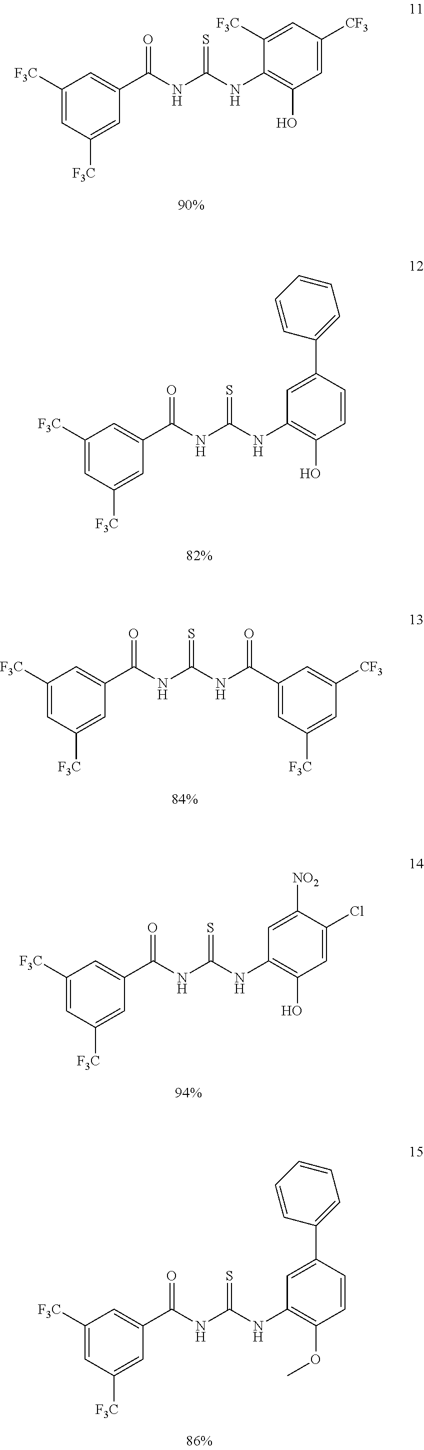 [SMILES: O=C(NC(=S)Nc1c(O)cc(C(F)(F)F)cc1C(F)(F)F)c1cc(C(F)(F)F)cc(C(F)(F)F)c1 ; O=C(NC(=S)Nc1cc(-c2ccccc2)ccc1O)c1cc(C(F)(F)F)cc(C(F)(F)F)c1 ; O=C(NC(=S)NC(=O)c1cc(C(F)(F)F)cc(C(F)(F)F)c1)c1cc(C(F)(F)F)cc(C(F)(F)F)c1 ; O=C(NC(=S)Nc1cc([N+](=O)[O-])c(Cl)cc1O)c1cc(C(F)(F)F)cc(C(F)(F)F)c1 ; COc1ccc(-c2ccccc2)cc1NC(=S)NC(=O)c1cc(C(F)(F)F)cc(C(F)(F)F)c1]

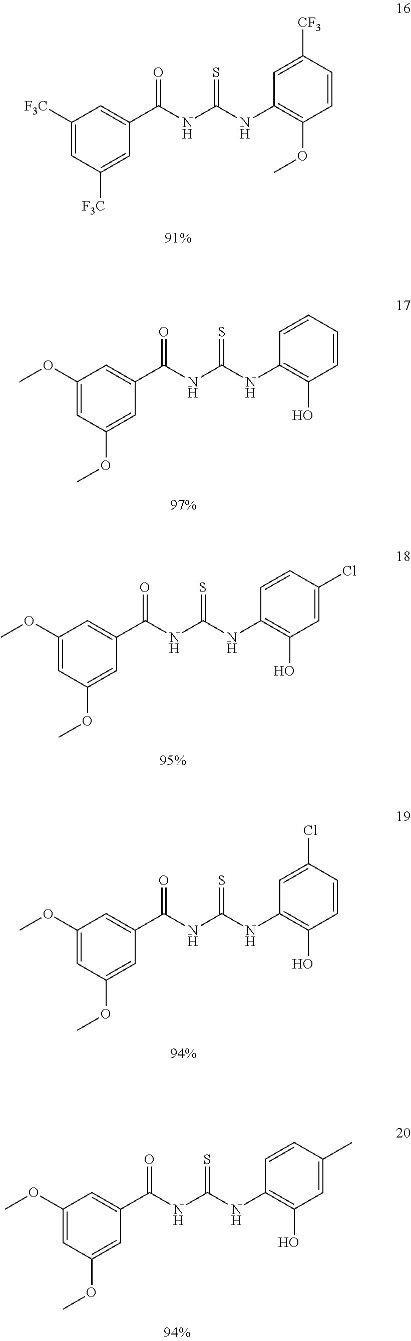

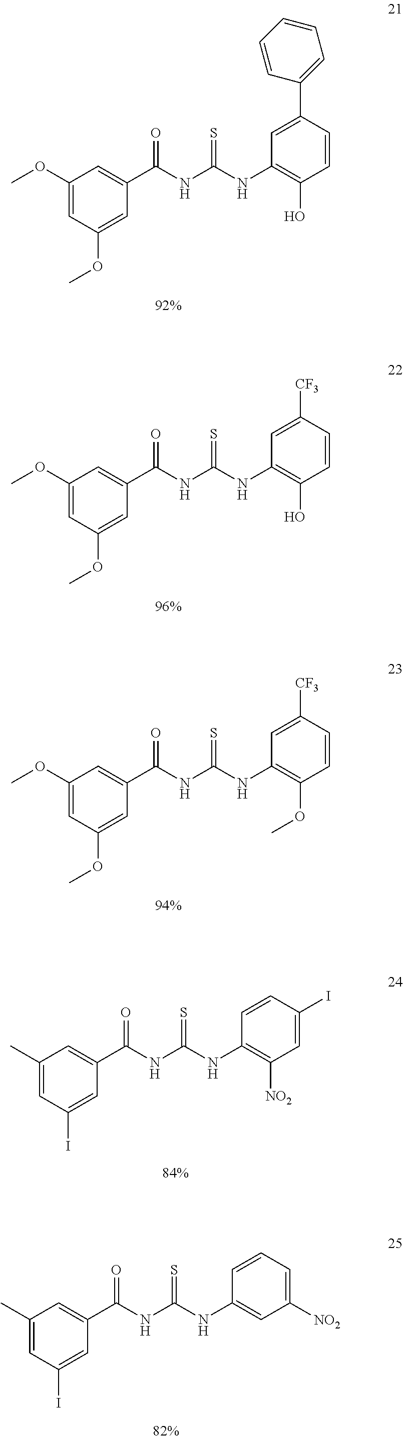 [SMILES: COc1cc(OC)cc(C(=O)NC(=S)Nc2cc(-c3ccccc3)ccc2O)c1 ; COc1cc(OC)cc(C(=O)NC(=S)Nc2cc(C(F)(F)F)ccc2O)c1 ; COc1cc(OC)cc(C(=O)NC(=S)Nc2cc(C(F)(F)F)ccc2OC)c1 ; Cc1cc(I)cc(C(=O)NC(=S)Nc2ccc(I)cc2[N+](=O)[O-])c1 ; Cc1cc(I)cc(C(=O)NC(=S)Nc2cccc([N+](=O)[O-])c2)c1]

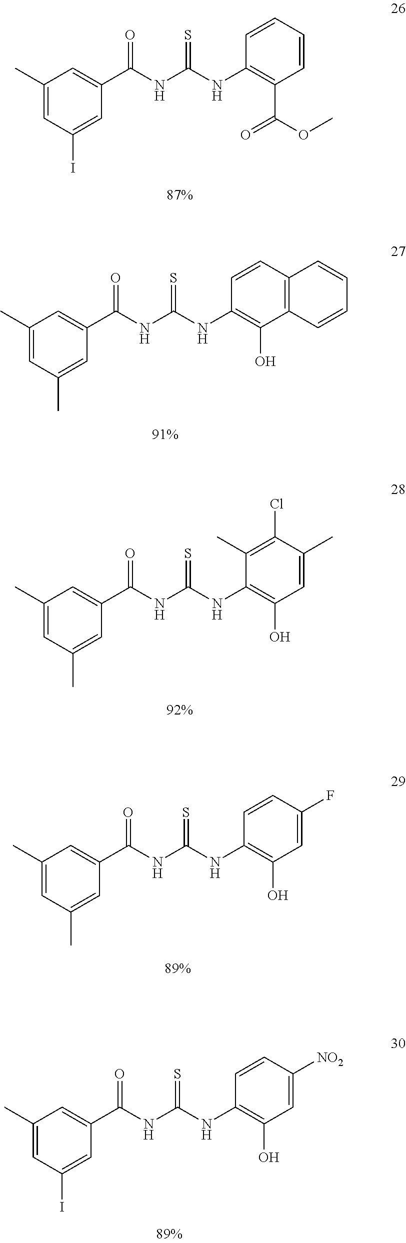

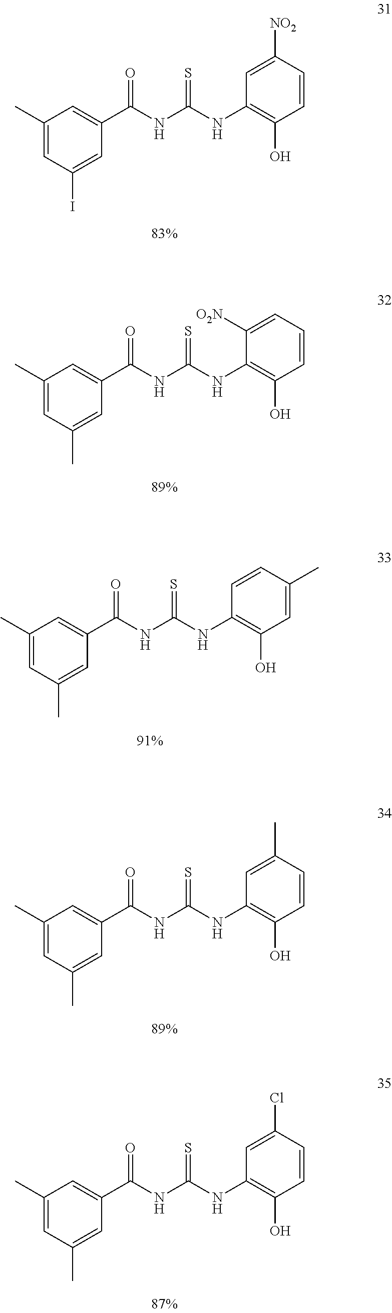

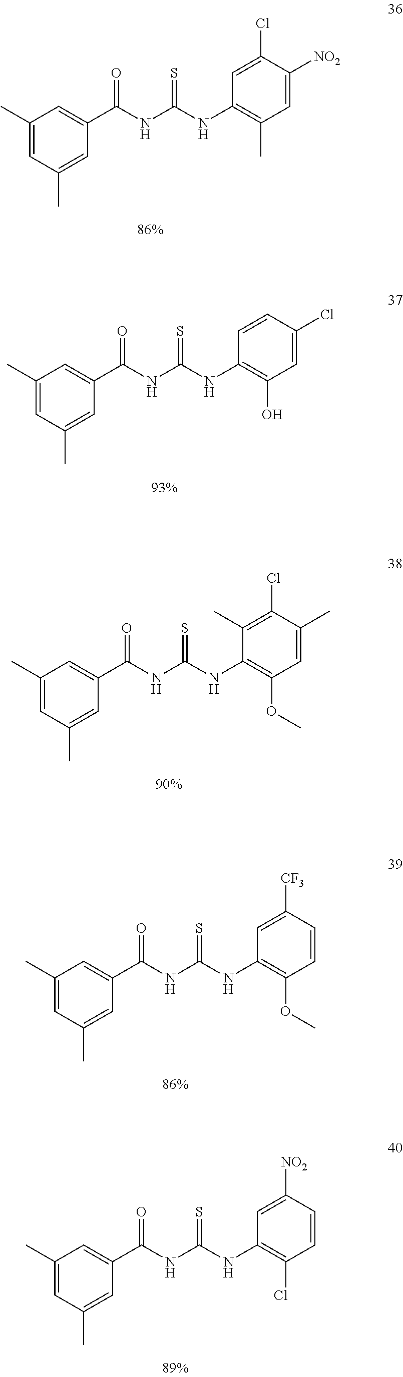

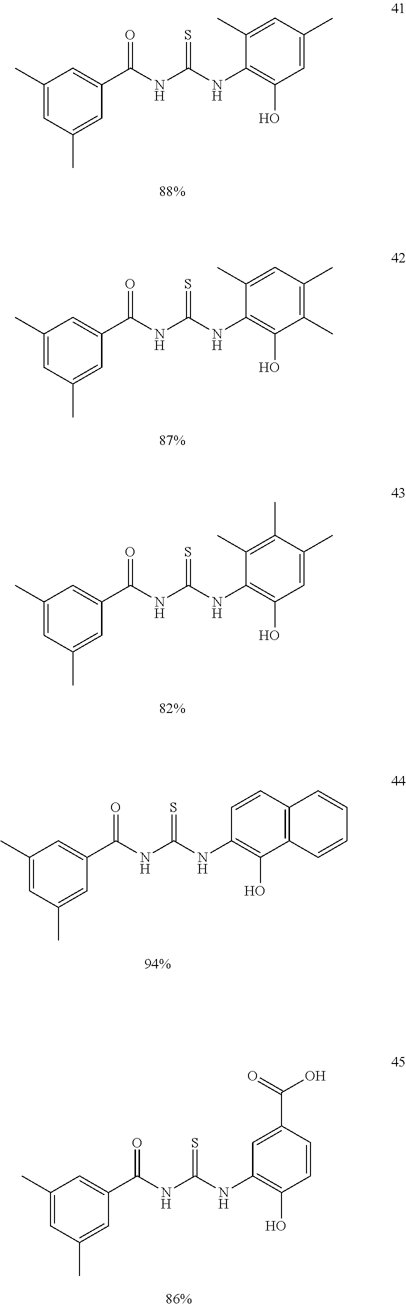

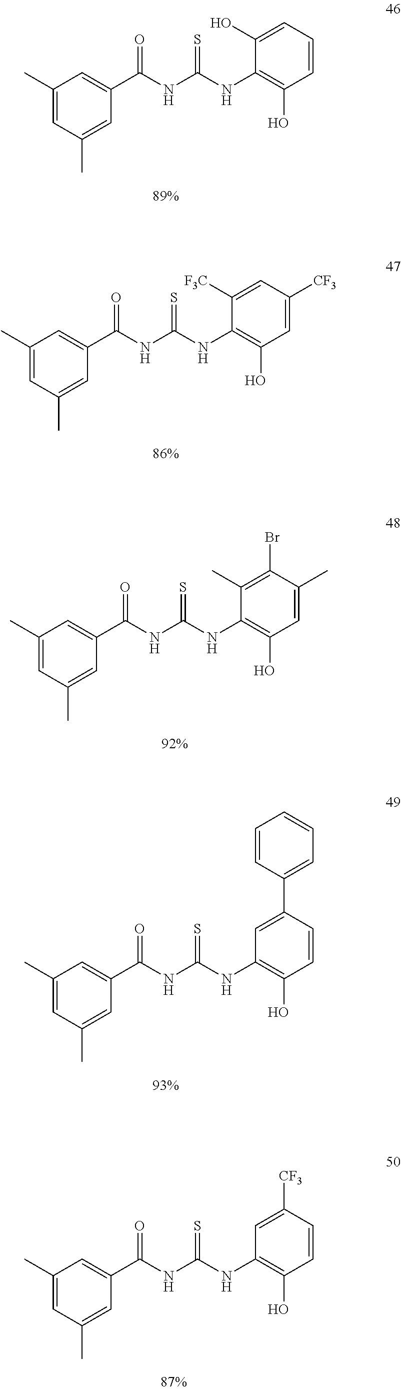

### Example 5

### Synthesis and Characterization of Various Nitro Compounds

### 1.1-iodo-2-nitroaniline (Nit01)

The general experimental procedure A was followed. Yellow solid. M. P.: 172.2° C.; IR (CHCl₃): ν 3490, 3377, 1610, 1555, 1460, 1376, 1336, 1231 cm-1; ¹H NMR (CDCl₃, 200 MHz): δ 6.11 (br s, 2H), 6.61 (d, J=8.7, Hz, 1H), 7.57 (dd, J=2.0, 8.7 Hz, 1H), 8.43 (d, J=2.0, Hz, 1H) ppm; ¹³C NMR (CDCl₃, 50 MHz): δ 75.8 (s), 120.6 (d), 132.9 (d), 134.2 (s), 143.7 (s), 144.0 (d) ppm; ESI-MS (m/z): 287.0 [M+Na]⁺; Anal. Calcd for C6H5IN2O2: C, 27.29; H, 1.91; N, 10.61. Found: C, 27.31; H, 1.93; N, 10.57.

### 1.2 2-nitronaphthalen-1-ol (Nit02)

The general experimental procedure A was followed. Yellow solid. M. P.: 117.6° C.; IR (CHCl₃): ν 3435, 3020, 1626, 1588, 1547, 1430, 1294, 1281, 1187, 1091, 1024, 669 cm-1; ¹H NMR (CDCl₃, 200 MHz): δ 7.34 (d, J=9.4, Hz, 1H), 5.62 (ddd, J=1.4, 6.8, 8.1 Hz, 1H), 7.72 (dt, J=1.3, 8.0 Hz, 1H), 7.82 (d, J=8.0, Hz, 1H), 8.01 (d, J=9.3 Hz, 1H), 8.52 (d, J=8.1 Hz, 1H), 12.25 (s, 1H) ppm; ¹³C NMR (CDCl₃, 50 MHz): δ 119.1 (d), 120.0 (d), 124.8 (s), 124.9 (d), 126.9 (d), 127.7 (d), 131.2 (d), 137.2 (s), 155.5 (s) ppm; ESI-MS (m/z): 221.4 [M+Na]⁺; Anal. Calcd for C₁₀H₇NO₃: C, 63.49; H, 3.73; N, 7.40 Found C, 68.49; H, 3.77; N, 7.45.

### 1.3 1-nitronaphthalen-2-ol (Nit03)

The general experimental procedure A was followed. Yellow solid.

M. P.: 160.1° C.; IR (CHCl₃): ν 3435, 1624, 1603, 1544, 1469, 1434, 1409, 1373, 1269, 1175, 1137, 827 cm-1; 1H NMR (CDCl₃, 200 MHz): δ 7.26 (d, J=9.1, Hz, 1H), 7.51 (ddd, J=1.1, 7.1, 8.1 Hz, 1H), 7.73 (ddd, J=1.5, 7.0, 8.6 Hz, 1H), 7.82 (dd, J=1.4, 8.0, Hz, 1H), 8.01 (d, J=9.1 Hz, 1H), 8.92 (dd, J=0.8, 8.8 Hz, 1H), 12.20 (s, 1H) ppm; 13C NMR (CDCl₃, 50 MHz): δ 119.2 (d), 123.1 (d), 125.5 (d), 126.7 (s), 128.5 (s), 129.2 (d), 130.8 (d), 139.1 (d), 158.7 (s) ppm; ESI-MS (m/z): 211.4 [M+Na]⁺; Anal. Calcd for C₁₀H₇NO₃: C, 63.49; H, 3.73; N, 7.40 Found: C, 63.53; H, 3.77; N, 7.45.

### 1.4 4-chloro-3,5-dimethyl-2-nitrophenol (Nit04)

The general experimental procedure B was followed.

Yellow solid. M. P.: 204.7° C.; IR (CHCl₃): ν 3368, 3291, 2506, 1637, 1598, 1560, 1520, 1459, 1377, 1350, 1105 cm-1; ¹H NMR (CDCl₃, 200 MHz): δ 2.41 (s, 3H), 2.62 (s, 3H), 6.93 (s, 1H), 9.59 (s, 1H) ppm; ¹³C NMR (CDCl₃, 50 MHz): δ 18.3 (q), 21.8 (q), 118.1 (d), 127.6 (s), 133.2 (s), 135.1 (s), 144.9 (s), 151.9 (s) ppm; ESI-MS (m/z): 223.3 [M+Na]⁺; Anal. Calcd for: C₈H₈ClNO₃: C, 47.66; H, 4.00; Cl, 17.59; N, 6.95. Found: C, 47.71; H, 4.06; Cl, 17.63; N, 7.00.

### 1.5 5-fluoro-2-nitrophenol (Nit05)

The general experimental procedure B was followed. Yellow solid. M. P.: 219.1° C.; IR (CHCl₃): ν 3409, 1659, 1599, 1565, 1460, 1375, 1218, 1101 cm-1; ¹H NMR (CDCl₃, 200 MHz): δ 6.73 (ddd, J=2.8, 7.5, 10.1 Hz, 1H), 6.85 (dd, J=2.7, 9.6 Hz, 1H), 8.17 (dd, J=5.8, 9.5 Hz, 1H), 10.81 (d, J=1.5, Hz, 1H) ppm; ¹³C NMR (CDCl₃, 50 MHz): δ 105.9 (d), 106.4 (d), 108.5 (d), 109.0 (d), 127.5 (d), 127.8 (d), 157.0 (s), 157.3 (s), 165.0 (s), 170.2 (s) ppm; ESI-MS (m/z): 179.2 [M+Na]⁺; Anal. Calcd for C₆H₄FNO₃: C, 45.87; H, 2.57; N, 8.92 Found: C, 45.93; H, 2.62; N, 8.89.

### 1.6 2-chloro-5-methoxy-1,3-dimethyl-4-nitrobenzene (Nit07)

The general experimental procedure B was followed.

Yellow solid. M. P.: 249.7° C.; IR (CHCl₃): ν 3020, 1596, 1559, 1533, 1464, 1329, 1109 cm-1; ¹H NMR (CDCl₃, 200 MHz): δ 2.31 (s, 3H), 2.42 (s, 3H), 3.86 (s, 3H), 6.78 (s, 1H) ppm; ¹³C NMR (CDCl₃, 50 MHz): δ 15.5 (q), 21.4 (q), 56.3 (q), 112.0 (d), 126.3 (s), 129.0 (s), 139.3 (s), 148.5 (s) ppm; ESI-MS (m/z): 237.3 [M+Na]⁺; Anal. Calcd for C₉H₁₀ClNO₃: C, 50.13; H, 4.67; N, 6.50 Found: C, 50.19; H, 4.72; N, 6.48.

### 1.7 1-methoxy-2-nitro-4-(trifluoromethyl)benzene (Nit08)

The general experimental procedure B was followed.

Yellow solid. M. P.: 210.1° C.; IR (CHCl₃): ν 3412, 3246, 1655, 1601, 1536, 11460, 1105 cm-1; ¹H NMR (CDCl₃, 200 MHz): δ 4.05 (s, 3H), 7.23 (d, J=8.8 Hz, 1H), 6.82 (ddd, J=0.6, 2.3, 8.8 Hz, 1H), 8.12 (d, J=1.9, Hz, 1H) ppm; ¹³C NMR (CDCl₃, 50 MHz): δ 56.9 (q), 82.0 (s), 113.9 (d), 118.5 (s), 123.3 (d), 128.0 (s), 131.1 (d), 155.2 (s) Ppm; ESI-MS (m/z): 243.2 [M+Na]⁺; Anal. Calcd for C₈H₆F₃NO₃: C, 43.45; H, 2.73; N, 6.33. Found: C, 43.50; H, 2.77; N, 6.29.

### 1.8 3,5-dimethyl-2-nitropheno (Nit09)

The general experimental procedure B was followed. Yellow solid. M. P.: 67.6° C.; IR (CHCl₃): ν 3022, 1984, 1619, 1586, 1540, 1463, 1347, 1287, 1215, 1064, 846 cm-1; ¹H NMR (CDCl₃, 200 MHz): δ 2.32 (s, 3H), 2.60 (s, 3H), 6.64 (s, 1H), 6.81 (s, 1H), 10.63 (s, 1H) ppm; ¹³C NMR (CDCl₃, 50 MHz): δ 21.4 (q), 22.5 (q), 117.4 (d), 125.2 (d), 132.8 (s), 136.6 (s), 147.1 (s), 155.6 (s) ppm; ESI-MS (m/z): 189.1 [M+Na]⁺; Anal. Calcd for C₈H₉NO₃: C, 57.48; H, 5.43; N, 8.38. Found: C, 57.53; H, 5.48; N, 8.40.

### 1.9 2,3,5-trimethyl-6-nitrophenol (Nit10)

The general experimental procedure B was followed.

Yellow solid. M. P.: 77.0-78.8° C.; IR (CHCl₃): ν 3683, 3371, 2934, 1607, 1581, 1458, 1400, 1340, 1277, 1215, 669 cm-1; ¹H NMR (CDCl₃, 200 MHz): δ 2.19 (s, 3H), 2.28 (s, 3H), 2.56 (s, 3H), 6.63 (s, 1H), 11.03 (s, 1H) ppm; ¹³C NMR (CDCl₃, 50 MHz): δ 11.6 (q), 20.4 (q), 22.4 (q), 124.4 (s), 125.1 (d), 133.0 (s), 133.1 (s), 145.4 (s), 153.8 (s) ppm; ESI-MS (m/z): 203.1 [M+Na]⁺; Anal. Calcd for C₉H₁₁NO₃: C, 59.66; H, 6.12; N, 7.73 Found: C, 59.72; H, 6.17; N, 7.69.

### 1.10 3,4,5-trimethyl-2-nitrophenol (Nit11)

The general experimental procedure B was followed.

Yellow solid. M. P.: 100.5° C.; IR (CHCl₃): ν 3408, 3021, 2928, 1614, 1579, 1527, 1467, 1338, 1264, 1215 cm-1; ¹H NMR (CDCl3, 200 MHz): δ 2.17 (s, 3H), 2.31 (s, 3H), 2.43 (s, 3H), 6.81 (s, 1H), 9.38 (s, 1H) ppm; ¹³C NMR (CDCl3, 50 MHz): δ 15.3 (q), 17.2 (q), 21.5 (q), 117.2 (d), 128.9 (s), 132.9 (s), 135.3 (s), 145.3 (s), 151.2 (s) Ppm; ESI-MS (m/z): 203.1 [M+Na]⁺; Anal. Calcd for C₉H₁₁NO₃: C, 59.66; H, 6.12; N, 7.73 Found: C, 59.72; H, 6.16; N, 7.68.

### 1.11 4-bromo-3,5-dimethyl-2-nitrophenol (Nit12)

The general experimental procedure B was followed. Yellow solid. M. P.: 105.0-107.0° C.; IR (CHCl₃): ν 3428, 3022, 2928, 1605, 1572, 1530, 1455, 1380, 1341, 1215, 1192, 756 cm-1; ¹H NMR (CDCl3, 200 MHz): δ 2.44 (s, 3H), 2.65 (s, 3H), 6.95 (s, 1H), 9.42 (br s, 1H) ppm; ¹³C NMR (CDCl3, 50 MHz): δ 21.8 (q), 25.0 (q), 118.2 (d), 120.0 (s), 135.0 (s), 146.7 (s), 152.2 (s) ppm; ESI-MS (m/z): 167.0 [M+Na]+; Anal. Calcd for C₈H₈BrNO₃: C, 39.05; H, 3.28; N, 5.69 Found: C, 38.98; H, 3.22; N, 5.61.

### 1.12 2-nitro-3,5-bis(trifluoromethyl)phenol (Nit13)

The general experimental procedure B was followed. Yellow solid. M. P.: 207.2-209.1° C.; IR (CHCl₃): ν 3430, 1700, 1616, 1552, 1447, 1386, 1275, 1216, 1153, 966 cm-1; ¹H NMR (CDCl3, 200 MHz): δ 7.58 (s, 1H), 7.65 (s, 1H) ppm; ¹³C NMR (CDCl3, 50 MHz): δ 115.4 (d), 115.5 (d), 115.5 (d), 115.6 (d), 118.7 (s), 119.5 (s), 120.2 (d), 120.3 (d), 124.2 (s), 124.9 (s), 125.3 (s), 126.0 (s), 134.4 (s), 135.1 (s) ppm; ESI-MS (m/z): 297.1 [M+Na]⁺; Anal. Calcd for C₈H₃F₆NO₃: C, 34.93; H, 1.10; F, 41.44; N, 5.09 Found: C, 34.89; H, 1.07; F, 41.39; N, 5.02.

### Example 6

### Synthesis and Characterization of Various Amino Compounds

### 2.1 1-aminonaphthalen-2-ol (AP01)

The general experimental procedure C was followed. Yellow solid. M. P.: 235.8° C.; IR (CHCl₃): ν 3385, 3313, 1633, 1604, 1573, 1508, 1463, 1376, 1336, 1274, 1072, 859 cm-1; ¹H NMR (CDCl₃, 200 MHz): δ 2.33 (s, 6H), 7.21 (s, 1H), 7.48 (s, 2H), 7.70 (s, 1H), 7.73 (d, J=8.8 Hz, 1H), 9.07 (d, J=8.8 Hz, 1H), 9.64 (s, 1H), 10.73 (s, 1H), 13.25 (s, 1H) ppm; ¹³C NMR (CDCl₃, 50 MHz): δ 116.9 (d), 119.1 (d), 119.6 (d), 122.3 (d), 124.5 (d), 124.8 (s), 125.8 (s), 127.7 (d), 128.5 (s), 140.2 (s) ppm; ESI-MS (m/z): 181.1 [M+Na]⁺; Anal. Calcd for C₁₀H₉NO: C, 75.45; H, 5.70; N, 8.80. Found: C, 75.50; H, 5.75; N, 8.85.

### 2.2, 2-amino-4-chloro-3,5-dimethylphenol (AP02)

The general experimental procedure C was followed. Yellow solid. M. P.: 147-148° C.; IR (CHCl₃): ν 3385, 3309, 1731, 1586, 1499, 1460, 1376, 1334, 1175 cm-1; ¹H NMR (CDCl₃, 200 MHz): δ 2.25 (s, 3H), 2.26 (s, 3H), 3.75 (br s, 2H), 6.52 (s, 1H) ppm; ¹³C NMR (CDCl₃, 50 MHz): δ 20.9 (q), 21.0 (q), 114.7 (d), 115.5 (d), 117.2 (d), 126.5 (d), 126.66 (d), 137.4 (s), 138.0 (s), 146.5 (s), 147.3 (s), 154.2 (s), 168.9 (s), 181.4 (s) ppm; ESI-MS (m/z): 193.1 [M+Na]⁺; Anal. Calcd for C₈H₁₀ClNO: C, 55.99; H, 5.87; Cl, 20.66; N, 8.16. Found: C, 56.05; H, 5.92; Cl, 20.72; N, 8.20.

### 2.3 2-aminobenzene-1,3-diol (AP03)

The general experimental procedure C was followed. Yellow solid. M. P.: 199.9° C.; IR (CHCl₃): 3389, 3300, 1624, 1604, 1525, 1464, 1377, 1308, 1147, 980 cm-1; ¹H NMR (CDCl₃, 200 MHz): δ 3.39 (br s, 2H), 6.28 (d, J=4.8 Hz, 1H), 6.30 (d, J=4.8 Hz, 2H), 8.64 (br s, 1H) ppm; ¹³C NMR (CDCl₃, 50 MHz): δ 106.8 (d), 116.6 (d), 123.3 (s), 145.1 (s) ppm; ESI-MS (m/z): 148.1 [M+Na]⁺; Anal. Calcd for C₆H₇NO₂: C, 57.59; H, 5.64; N, 11.19 Found: C, 57.63; H, 5.61; N, 11.14.

### 2.4 2-amino-4-(trifluoromethyl)phenol (AP04)

The general experimental procedure C was followed. Yellow solid. M. P.: 121° C.; IR (CHCl₃): ν 3386, 3311, 1618, 1528, 1461, 1376, 1331, 1165, 1098 cm-1; ¹H NMR (CDCl₃, 200 MHz): δ 3.35-4.78 (br s, 2H), 6.76 (d, J=8.1 Hz, 1H), 6.94 (ddd, J=0.8, 2.1, 8.1 Hz, 1H), 6.98 (d, J=1.9, Hz, 1H) ppm; ¹³C NMR (CDCl₃, 50 MHz): δ 19.7 (q), 114.6 (d), 120.8 (d), 121.0 (s), 124.2 (d), 124.8 (d), 126.0 (s), 130.5 (s), 133.3 (d), 136.6 (s), 146.2 (s), 166.8 (s), 176.1 (s) ppm; ESI-MS (m/z): 357.7 [M+Na]⁺; Anal. Calcd for C₇H₆F₃NO: C, 47.47; H, 3.41; N, 7.91 Found: C, 47.51; H, 3.45; N, 7.87.

### 2.5 2-methoxy-5-(trifluoromethyl)aniline (AP05)

The general experimental procedure C was followed. Yellow solid. M. P.: 208.6° C.; IR (CHCl₃): ν 3425, 3392, 1740, 1652, 1567, 1374, 1233, 1144 cm-1; ¹H NMR (CDCl₃, 200 MHz): δ 3.89 (s, 3H), 3.94 (br s, 2H), 6.81 (d, J=8.3 Hz, 1H), 7.91 (d, J=2.0, Hz, 1H), 6.99 (ddd, J=0.8, 2.1, 8.3 Hz, 1H) ppm; ¹³C NMR (CDCl₃, 50 MHz): δ 55.5 (q), 109.5 (d), 110.8 (d), 110.9 (d), 111.0 (d), 111.1 (d), 115.3 (d), 115.4 (d), 115.5 (d), 115.6 (d), 121.8 (s), 122.7 (s), 123.4 (s), 124.0 (s), 127.2 (s), 136.4 (s), 149.3 (s) ppm; ESI-MS (m/z): 213.2 [M+Na]⁺; Anal. Calcd for C₈H₈F3NO: C, 50.27; H, 4.22; N, 7.33. Found: C, 50.31; H, 4.27; N, 7.29.

### 2.6 2-amino-3,5-dimethylphenol (AP06)

The general experimental procedure C was followed. Yellow solid. M. P.: 159.6° C.; IR (CHCl₃): ν 3376, 3308, 2921, 2664, 1599, 1516, 1462, 1376, 1304, 840 cm-1; ¹H NMR (CDCl₃, 200 MHz): δ 2.16 (s, 3H), 2.18 (s, 3H), 3.53 (br s, 2H), 6.44 (s, 1H), 6.51 (s, 1H) ppm; ¹³C NMR (CDCl3, 50 MHz): δ 16.2 (q), 19.6 (q), 112.4 (d), 120.8 (d), 121.8 (s), 125.5 (s), 129.7 (s), 143.3 (s) ppm; ESI-MS (m/z): 159.6 [M+Na]⁺; Anal. Calcd for C₈H₁₁NO: C, 70.04; H, 8.08; N, 10.21 Found: C, 70.10; H, 8.11; N, 10.17.

### 2.7 2-amino-3,5,6-trimethylphenol (AP07)

The general experimental procedure C was followed. Yellow solid. M. P.: 151.0-153.0° C.; IR (CHCl₃): ν 3400, 3317, 2853, 1577, 1496, 1463, 1377, 1327, 1161, 847 cm-1; ¹H NMR (CDCl₃, 200 MHz): δ 2.12 (s, 3H), 2.16 (s, 3H), 2.17 (s, 3H), 3.68 (br s, 2H), 6.53 (s, 1H) ppm; ¹³C NMR (CDCl₃, 50 MHz): δ 10.5 (q), 15.4 (q), 17.5 (q), 117.3 (s), 119.1 (s), 121.1 (d), 122.4 (s), 130.9 (s), 139.9 (s) ppm; ESI-MS (m/z): 173.2 [M+Na]⁺; Anal. Calcd for C₉H₁₃NO: C, 71.49; H, 8.67; N, 9.26 Found: C, 71.53; H, 8.72; N, 9.30.

### 2.8 2-amino-3,4,5-trimethylphenol (AP08)

The general experimental procedure C was followed. Yellow solid. M. P.: 158.4° C.; IR (CHCl₃): ν 3376, 3310, 2853, 2668, 1605, 1464, 1376, 1333, 1290, 920 cm-1; ¹H NMR (CDCl₃, 200 MHz): δ 2.11 (s, 3H), 2.12 (s, 3H), 2.15 (s, 3H), 3.65 (br s, 2H), 6.45 (s, 1H) ppm; ¹³C NMR (CDCl3, 50 MHz): δ 13.4 (q), 15.0 (q), 19.8 (q), 113.4 (d), 120.9 (s), 123.4 (s), 124.4 (s), 131.6 (s), 141.5 (s) ppm; ESI-MS (m/z): 173.1 [M+Na]⁺; Anal. Calcd for C₉H₁₃NO: C, 71.49; H, 8.67; N, 9.26 Found: C, 71.53; H, 8.70; N, 9.22.

### 2.9 2-amino-4-bromo-3,5-dimethylphenol (AP09)

The general experimental procedure C was followed. Yellow solid. M. P.: 121.0-122.0° C.; IR (CHCl₃): ν 3382, 3306, 2853, 1708, 1587, 1459, 1376, 1332, 1155, 1021 cm-1; ¹H NMR (CDCl₃, 200 MHz): δ 3382, 3306, 2853, 1708, 1587, 1459, 1376, 1332, 1155, 1021 ppm; ¹³C NMR (CDCl3, 50 MHz): δ 17.2 (q), 20.5 (q), 118.4 (s), 123.1 (d), 125.3 (s), 129.0 (s), 131.1 (s), 144.5 (s) ppm; ESI-MS (m/z): 137.0 [M+Na]⁺; Anal. Calcd for C₈H₁₀BrNO: C, 44.47; H, 4.66; N, 6.48 Found: C, 44.51; H, 4.70; N, 6.51.

### 2.10 2-amino-3,5-bis(trifluoromethyl)phenol (AP10)

The general experimental procedure C was followed. Yellow solid. M. P.: 167.5° C.; IR (CHCl₃): ν 3404, 329, 1637, 1552, 1456, 1383, 1268, 1171, 942 cm-1; ¹H NMR (CDCl₃, 200 MHz): δ 4.67 (br s, 2H), 6.15 (br s, 1H), 7.06 (d, J=1.4 Hz, 1H), 7.32 (br s, 1H) ppm; ¹³C NMR (CDCl₃, 50 MHz): 113.9 (d), 115.9 (d), 115.9 (d), 119.0 (s), 119.7 (s), 121.3 (s), 121.5 (s), 126.6 (s), 127.0 (s), 137.0 (s), 144.0 (s) ppm; ESI-MS (m/z): 267.1 [M+Na]⁺; Anal. Calcd for C₈H₅F₆NO: C, 39.20; H, 2.06; N, 5.71 Found: C, 39.16; H, 2.00; N, 5.68.

### Example 7

### Synthesis and Characterization of Various Thiourea Compounds

### 3.1 N-(2-hydroxyphenylcarbamothioyl)-3,5-bis(trifluoromethyl)benzamide (TU01)

The general procedure D was followed. Ammoniumthiocyanate (55 mg, 0.72 mmol) 3,5-bis(trifluromethyl)benzoyl chloride (0.36 mmol) 2-aminophenol (39.5 mg, 0.36 mmol) were used to obtain 1 (136 mg, 92%) as yellow solid.

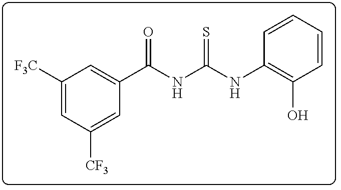 [SMILES: O=C(NC(=S)Nc1ccccc1O)c1cc(C(F)(F)F)cc(C(F)(F)F)c1]

M. P.: 175° C.; IR (CHCl₃): ν 3584, 3362, 3020, 1675, 1608, 1542, 1457, 1280, 1215, 1149, 1123, 754 cm⁻¹; ¹H NMR (CDCl₃, −200 MHz): δ 6.30 (s, 1H), 7.02 (dd, J=1.4, 9.5 Hz, 1H), 7.08 (d, J=1.9 Hz, 1H), 7.28 (ddd, J=1.5, 7.4, 15.5 Hz, 1H), 7.65 (dd, J=1.58, 8.01 Hz, 1H), 8.16 (s, 1H), 8.39 (s, 2H), 9.58 (s, 1H), 12.45 (s, 1H) ppm; ¹³C NMR (CDCl₃, 50 MHz): δ 119.26 (s), 121.61 (s), 124.99 (s, 2C), 125.54 (s), 127.10 (d), 128.06 (d, 2C), 129.11 (s, 2C), 132.73 (s), 133.42 (s), 133.73 (s), 149.27 (s), 164.57 (s), 177.00 (s) ppm; EI-MS (m/z): 408.04 [M⁺].

### 3.2 N-(2-hydroxy-5-nitrophenylcarbamothioyl)-3,5-bis(trifluoromethyl)benzamide (TU02)

The general procedure D was followed. Ammoniumthiocyanate (55 mg, 0.72 mmol) 3,5-bis(trifluromethyl)benzoyl chloride (0.36 mmol) 2-amino-4-nitro phenol (55 mg, 0.36 mmol) were used to obtain 2 (148 mg, 90%) as yellow solid.

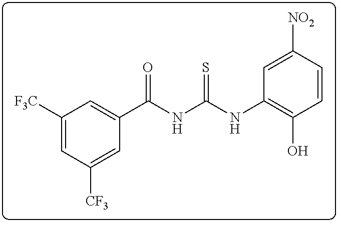 [SMILES: O=C(NC(=S)Nc1cc([N+](=O)[O-])ccc1O)c1cc(C(F)(F)F)cc(C(F)(F)F)c1]

M. P.: 132° C.; IR (CHCl₃): ν 3503, 3020, 2400, 1700, 1602, 1542, 1474, 1216, 770, 669 cm⁻¹; ¹H NMR (CDCl₃+DMSO-d₆, 200 MHz): δ 7.09 (d, J=8.97 Hz, 1H), 8.00 (dd, J=2.74, 8.97 Hz, 1H), 8.08 (s, 1H), 8.64 (s, 2H), 9.89 (d, J=2.76 Hz, 1H), 11.25 (s, 1H), 11.92 (s, 1H), 13.14 (s, 1H) ppm; ¹³C NMR (CDCl₃, 50 MHz): δ 114.38 (s) 118.11 (s), 122.10 (s), 125.31 (s), 125.8 (t, 2C), 126.11 (s, 2C), 129.2 (d, 2C), 131.07 (s), 131.75 (s), 134.05 (s), 139.13 (s), 154.62 (s), 165.26 (s), 177.55 (s) ppm; EI-MS (m/z): 453.0 [M+]; HRMS ESI calcd for (MH⁺) C₁₆H₁₀N₃O₄F₆S 454.0291. found 454.0291.

### 3.3 N-(2-hydroxy-4-nitrophenylcarbamothioyl)-3, 5-bis(trifluoromethyl)benzamide (TU03)

The general procedure D was followed. ammonium thiocyanate (55 mg, 0.72 mmol) 3,5-bis(trifluromethyl)benzoyl chloride (0.36 mmol) 2-amino-5-nitro phenol (55 mg, 0.36 mmol) were used to obtain 3 (152 mg, 94%) as yellow solid.

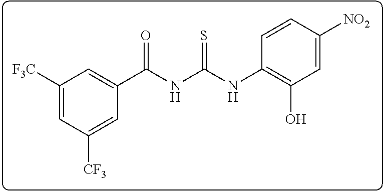 [SMILES: O=C(NC(=S)Nc1ccc([N+](=O)[O-])cc1O)c1cc(C(F)(F)F)cc(C(F)(F)F)c1]

M. P.: 187° C.; IR (CHCl₃): ν 3309, 3020, 1661, 1616, 1555, 1525, 1456, 1280, 1215, 1146, 1123, 756 cm⁻¹; ¹H NMR (CDCl₃+DMSO-d₆, 200 MHz): δ 7.77 (dd, J=2.55, 9.07 Hz, 1H), 7.86 (d, J=2.5, Hz, 1H), 8.08 (s, 1H), 8.65 (s, 2H), 9.20 (d, J=9.07 Hz, 1H), 10.91 (s, 1H), 12.10 (s, 1H), 13.34 (s, 1H) ppm; ¹³C NMR (CDCl₃+DMSO-d₆, 50 MHz): δ 109.18 (s) 113.89 (s), 120.70 (s), 125.6 (t, 2C), 129.1 (d, 2C), 130.73 (s), 131.47 (s), 132.17 (s, 2C), 133.81 (s), 144.03 (s), 148.29 (s), 165.02 (s), 177.37 (s) ppm; EI-MS (m/z): 453.0 [M+]; HRMS ESI calcd for (MH⁺) C₁₆H₁₀N₃O₄F₆S 454.0291. found 454.0291.

### 3.4 N-(4-chloro-2-hydroxyphenylcarbamothioyl)-3,5-bis(trifluoromethyl)benzamide (TU04)

The general procedure D was followed. ammoniumthiocyanate (55 mg, 0.72 mmol) 3,5-bis(trifluromethyl)benzoyl chloride (0.36 mmol) 2-amino-5-chloro phenol (52 mg, 0.36 mmol) were used to obtain 4 (138 mg, 86%) as yellow solid.

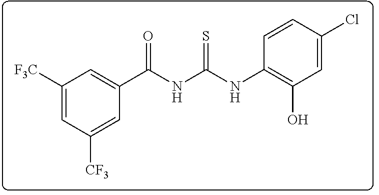 [SMILES: O=C(NC(=S)Nc1ccc(Cl)cc1O)c1cc(C(F)(F)F)cc(C(F)(F)F)c1]

M. P.: 156° C.; IR (CHCl₃): ν 3546, 3362, 3020, 1672, 1601, 1562, 1541, 1421, 1278, 1216, 1128, 757, 681 cm⁻¹; ¹H NMR (CDCl₃, 200 MHz): δ 7.02 (dd, J=2.1, 8.5 Hz, 1H), 7.08 (d, J=2.1 Hz, 1H), 7.59 (d, J=8.5 Hz, 1H), 8.18 (s, 1H), 8.35 (s, 2H), 9.34 (s, 1H), 12.40 (s, 1H) ppm; ¹³C NMR (CDCl₃+DMSO-d₆, 50 MHz): δ 115.5 (s) 118.6 (s), 119.9 (s), 123.8 (s), 124.7 (s), 125.8 (t), 129.1 (d), 131.1 (s), 131.8 (s), 134.2 (s), 149.7 (s), 165.1 (s), 177.1 (s) ppm; EI-MS (m/z): 442.0 [M+]; HRMS ESI calcd for (MH⁺) C₁₆H₁₀N₂O₂ClF₆S 443.0056. found 443.0050.

### 3.5 N-(5-chloro-2-hydroxyphenylcarbamothioyl)-3,5-bis(trifluoromethyl)benzamide (TU05)

The general procedure D was followed. Ammonium thiocyanate (55 mg, 0.72 mmol) 3,5-bis(trifluromethyl)benzoyl chloride (0.36 mmol) 2-amino-4-chloro phenol (52 mg, 0.36 mmol) were used to obtain 5 (144 mg, 90%) as yellow solid.

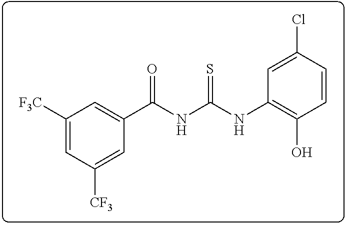 [SMILES: O=C(NC(=S)Nc1cc(Cl)ccc1O)c1cc(C(F)(F)F)cc(C(F)(F)F)c1]

M. P.: 156° C.; IR (CHCl₃): ν (3362, 3020, 1734, 1674, 1542, 1496, 1348, 1278, 1185, 1139, 761, 681 cm⁻¹; ¹H NMR (CDCl₃, 400 MHz): δ 7.09 (d, J=8.97 Hz, 1H), 8.00 (dd, J=2.74, 8.97 Hz, 1H), 8.08 (s, 1H), 8.64 (s, 2H), 9.89 (d, J=2.76 Hz, 1H), 11.25 (s, 1H), 11.92 (s, 1H), 13.14 (s, 1H) ppm; ¹³C NMR (CDCl₃, 125 MHz): δ 119.47 (s) 121.14 (s), 123.85 (s), 124.48 (s), 125.85 (s), 127.2 (d), 128.18 (s), 128.54 (s), 133.08 (s), 133.52 (s), 147.74 (s), 164.77 (s), 177.06 (s) ppm; EI-MS (m/z): 442.0 [M+]; HRMS ESI calcd for (MH⁺) C₁₆H₁₀N₂O₂ClF₆S 443.0056. found 443.0050.

### 3.6 N-(2-hydroxy-5-methylphenylcarbamothioyl)-3,5-bis(trifluoromethyl)benzamide (TU06)

The general procedure D was followed Ammonium thiocyanate (55 mg, 0.72 mmol) 3,5-bis(trifluromethyl)benzoyl chloride (0.36 mmol) 2-amino-4-methyl phenol (44 mg, 0.36 mmol) were used to obtain 6 (136 mg, 89%) as yellow solid.

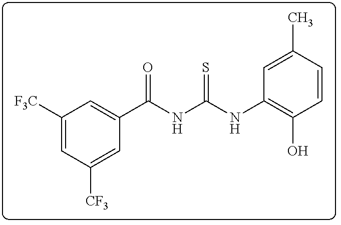 [SMILES: Cc1ccc(O)c(NC(=S)NC(=O)c2cc(C(F)(F)F)cc(C(F)(F)F)c2)c1]

M. P.: 149° C.; IR (CHCl₃): ν 3588, 3362, 3020, 2926, 1685, 1604, 1560, 1542, 1457, 1351, 1279, 1215, 1141, 756, 681 cm⁻¹; ¹H NMR (CDCl₃, 200 MHz): δ 2.32 (s, 3H), 6.19 (s, 1H), 6.95 (d, J=8.2 Hz, 1H), 7.07 (dd, J=1.7, 8.3 Hz, 1H), 7.36 (d, J=1.7 Hz, 1H), 8.15 (s, 1H), 8.37 (s, 2H), 9.54 (s, 1H), 12.38 (s, 1H) ppm; ¹³C NMR (CDCl₃, 50 MHz): δ 20.5 (s), 119.2 (s), 125.1 (s), 125.2 (s), 127.1 (t), 128.0 (d), 129.8 (s), 131.3 (s), 132.6 (s), 133.3 (s), 133.7 (s), 147.0 (s), 164.6 (s), 176.9 (s) ppm; EI-MS (m/z): 422.1 [M+].

### 3.7 N-(2-hydroxy-4-methylphenylcarbamothioyl)-3, 5-bis(trifluoromethyl)benzamide (TU07)

The general procedure D was followed. Ammonium thiocyanate (55 mg, 0.72 mmol) 3,5-bis(trifluromethyl)benzoyl chloride (0.36 mmol) 2-amino-5-methyl phenol (44 mg, 0.36 mmol) were used to obtain 7 (139 mg, 91%) as yellow solid.

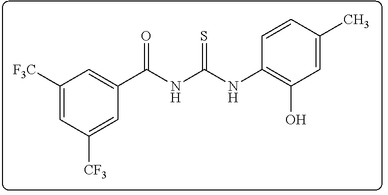 [SMILES: Cc1ccc(NC(=S)NC(=O)c2cc(C(F)(F)F)cc(C(F)(F)F)c2)c(O)c1]

M. P.: 149° C.; IR (CHCl₃): ν 3588, 3362, 3020, 2926, 1685, 1604, 1560, 1542, 1457, 1351, 1279, 1215, 1141, 756, 681 cm⁻¹; ¹H NMR (CDCl₃, 200 MHz): δ 2.35 (s, 3H), 6.18 (s, 1H), 6.85 (dd, J=1.1, 8.0 Hz, 1H), 6.89 (d, J=1.1 Hz, 1H), 7.44 (d, J=8.0 Hz, 1H), 8.16 (s, 1H), 8.37 (s, 2H), 9.41 (s, 1H), 12.34 (s, 1H) ppm; ¹³C NMR (CDCl₃+DMSO-d₆, 50 MHz): δ 20.2 (s), 115.1 (s), 118.4 (s), 122.4 (s), 122.5 (s), 124.8 (q), 128.6 (d), 130.1 (s), 130.7 (s), 133.7 (s), 135.8 (s), 148.1 (s), 164.4 (s), 176.2 (s) ppm; EI-MS (m/z): 422.1 [M+].

### 3.8 N-(2-hydroxy-3-nitrophenylcarbamothioyl)-3,5-bis(trifluoromethyl)benzamide (TU08)

The general procedure D was followed. ammonium thiocyanate (55 mg, 0.72 mmol) 3,5-bis(trifluromethyl)benzoyl chloride (0.36 mmol) 2-amino-6-nitro phenol (55 mg, 0.36 mmol) were used to obtain 8 (144 mg, 88%) as yellow solid.

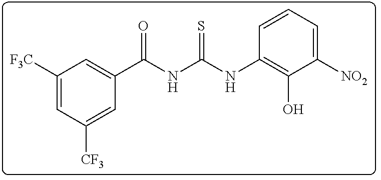 [SMILES: O=C(NC(=S)Nc1cccc([N+](=O)[O-])c1O)c1cc(C(F)(F)F)cc(C(F)(F)F)c1]

M. P.: 147° C.; IR (CHCl₃): ν 3393, 3020, 1688, 1618, 1541, 1524, 1456, 1280, 1215, 1146, 756 cm⁻¹; ¹H NMR (CDCl₃+DMSO-d₆, 500 MHz): δ 7.33 (d, J=2.4 Hz, 1H), 7.51 (dd, J=3.6, 6.0 Hz, 1H), 8.08 (s, 1H), 8.58 (d, J=8.7 Hz, 1H), 8.66 (s, 2H), 9.95 (s, 1H), 12.22 (s, 1H), 12.37 (s, 1H) ppm; ¹³C NMR (CDCl₃+DMSO-d₆, 125 MHz): δ 115.00 (s) 118.85 (s), 121.60 (s), 125.6 (t, 2C), 127.77 (s), 127.97 (d, 2C), 129.18 (d), 130.99 (s, 2C), 131.26 (s), 133.90 (s), 146.42 (s), 153.61 (s), 165.48 (s), 180.71 (s) ppm; EI-MS (m/z): 453.0 [M+].

### 3.9 N-(2-hydroxy-5-(trifluromethyl)phenylcarbamothioyl)-3,5-bis(trifluoromethyl)benzamide (TU09)

The general procedure D was followed. ammonium thiocyanate (55 mg, 0.72 mmol) 3,5-bis(trifluromethyl)benzoyl chloride (0.36 mmol) 2-amino-4-(trifluoromethyl)phenol (64 mg, 0.36 mmol) were used to obtain 9 (154 mg, 90%) as yellow solid.

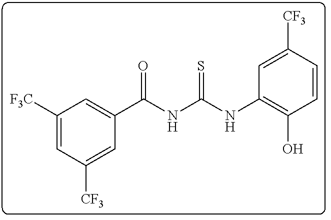 [SMILES: O=C(NC(=S)Nc1cc(C(F)(F)F)ccc1O)c1cc(C(F)(F)F)cc(C(F)(F)F)c1]

M. P.: 176° C.; IR (CHCl₃): ν 3402, 3021, 1682, 1615, 1514, 1332, 1280, 1216, 1149, 1084123, 772 cm⁻¹; ¹H NMR (CDCl₃, 200 MHz): δ 6.63 (br s, 1H), 7.15 (d, J=8.6 Hz, 1H), 7.54 (dd, J=2.1, 8.6 Hz, 1H), 7.54 (d, J=2.1 Hz, 1H), 8.18 (s, 1H), 8.38 (s, 2H), 9.41 (s, 1H), 12.54 (s, 1H) ppm; ¹³C NMR (CDCl₃, 50 MHz): δ21.3 (d, 2C), 84.5 (s), 123.6 (d), 125.1 (d, 2C), 133.6 (s), 134.0 (d), 134.5 (d), 135.1 (s), 136.5 (s), 138.8 (s, 2C), 144.6 (d), 165.9 (s), 177.8 (s) ppm; HRMS ESI calcd for (MH⁺) C₁₇H₁₀N₂O₂F₉S 477.0314. found 477.0314.

### 3.10 N-(3-bromo-6-hydroxy-2,4-dimethylphenylcarbamothioyl)-3,5bis(trifluoromethyl)benzamide (TU10)

The general procedure D was followed. ammoniumthiocyanate (55 mg, 0.72 mmol) 3,5-bis(trifluromethyl)benzoyl chloride (0.36 mmol) 2-amino-4-bromo-3,5-dimethylphenol (78 mg, 0.36 mmol) were used to obtain 10 (172 mg, 91%) as yellow solid.

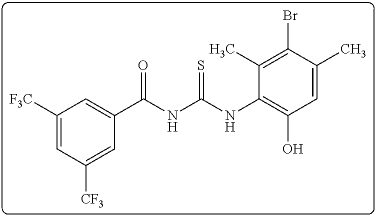 [SMILES: Cc1cc(O)c(NC(=S)NC(=O)c2cc(C(F)(F)F)cc(C(F)(F)F)c2)c(C)c1Br]

M. P.: 167° C.; IR (CHCl₃): ν 3403, 3020, 1686, 1580, 1542, 1523, 1458, 1379, 1280, 1216, 1188, 1148, 909, 757 cm⁻¹; ¹H NMR (CDCl₃, 200 MHz): δ 2.40 (s, 3H), 2.43 (s, 3H), 6.89 (br s, 1H), 8.18 (s, 1H), 8.40 (s, 2H), 7.56 (s, 2H), 9.68 (s, 1H), 12.0 (s, 1H) ppm; ¹³C NMR (CDCl₃, 50 MHz): δ19.4 (s), 24.1 (s), 118.5 (s), 119.3 (s), 122.9 (s), 127.2 (q, 2C), 128.1 (d, 2C), 132.7 (s), 133.5 (s), 134.5 (s), 140.1 (s), 149.5 (s), 164.8 (s), 178.8 (s) ppm; HRMS ESI calcd for (MH⁺) C₁₈H₁₄N₂O₂⁸¹BrF₆S 516.9838. found 516.9838, calcd for [M+Na]⁺ C₁₈H₁₃N₂O₂⁸¹BrF₆SNa 538.9658. found 538.9657.

### 3.11 N-(2-hydroxy-4,6-bis(trifluromethyl)phenylcarbamothioyl)-3,5bis(trifluoromethyl)benzamide (TU11)

The general procedure D was followed. ammoniumthiocyanate (55 mg, 0.72 mmol) 3,5-bis(trifluromethyl)benzoyl chloride (0.36 mmol) 2-amino-3,5-bis(trifluoromethyl)phenol (88 mg, 0.36 mmol) were used to obtain 11 (176 mg, 90%) as yellow solid.

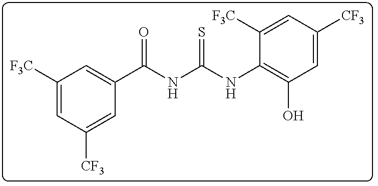 [SMILES: O=C(NC(=S)Nc1c(O)cc(C(F)(F)F)cc1C(F)(F)F)c1cc(C(F)(F)F)cc(C(F)(F)F)c1]

M. P.: 147° C.; IR (CHCl₃): ν 3584, 3362, 3019, 1700, 1675, 1583, 1509, 1280, 1219, 1149, 1123, 772 cm⁻¹; ¹H NMR (CDCl₃, 200 MHz): δ 7.40 (br s, 1H), 8.14 (s, 1H), 8.34 (s, 2H), 8.43 (s, 1H), 9.47 (s, 1H), 9.96 (s, 1H), 12.52 (s, 1H) ppm; ¹³C NMR (CDCl₃, 50 MHz): δ 119.8 (s), 120.2 (s), 125.2 (s, 2C), 127.0 (t, 2C), 128.0 (d, 3C), 131.6 (s, 2C), 133.3 (s), 133.8 (s), (d, 2C), 84.5 (s), 123.6 (d), 125.1 (d, 2C), 133.6 (s), 134.0 (d), 134.5 (d), 135.1 (s), 136.5 (s), 134.9 (s), 137.2 (s), 150.4 (s), 163.9 (s), 181.9 (s) ppm; HRMS ESI calcd for (MH⁺) C₁₈H₉N₂O₂F₁₂S 545.0193. found 545.0194.

### 3.12 N-((4-hydroxy-[1,1′-biphenyl]-3-yl)carbamothioyl)-3,5-bis(trifluoromethyl)benzamide (TU12)

The general procedure D was followed ammoniumthiocyanate (55 mg, 0.72 mmol) 3,5-bis(trifluromethyl)benzoyl chloride (0.36 mmol) 3-aminobiphenyl-4-ol (64 mg, 0.36 mmol) were used to obtain 12 (142 mg, 82%) as yellow solid.

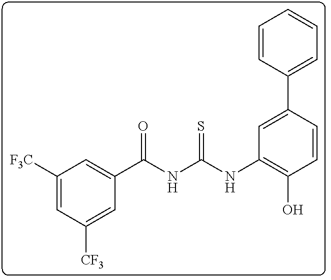 [SMILES: O=C(NC(=S)Nc1cc(-c2ccccc2)ccc1O)c1cc(C(F)(F)F)cc(C(F)(F)F)c1]

M. P.: 173° C.; IR (CHCl₃): ν 3423, 2926, 2854, 1690, 1610, 1559, 1474, 1326, 1279, 1243, 1183, 1136, 976, 761 cm⁻¹; ¹H NMR (CDCl₃, 200 MHz): δ 7.16 (d, J=8.4 Hz, 1H), 7.34-7.38 (m, 1H), 7.44-7.54 (m, 5H) 7.94 (d, J=2.2 Hz, 1H), 8.17 (s, 1H), 8.40 (s, 2H), 9.51 (s, 1H), 12.56 (s, 1H) ppm; HRMS ESI calcd for (MH⁺) C₂₂H₁₅N₂O₂F₆S 485.0758. found 485.0760.

### 3.13 N,N′-thiocarbonylbis(3,5-bis(trifluoromethyl)benzamide)(TU13)

The general procedure D was followed. ammoniumthiocyanate (55 mg, 0.72 mmol) 3,5-bis(trifluromethyl)benzoyl chloride (0.36 mmol) 3,5-bis(trifluromethyl)benzamide (93 mg, 0.36 mmol) were used to obtain 13 (168 mg, 84%) as yellow solid.

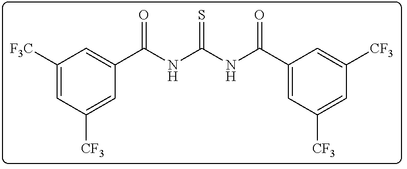 [SMILES: O=C(NC(=S)NC(=O)c1cc(C(F)(F)F)cc(C(F)(F)F)c1)c1cc(C(F)(F)F)cc(C(F)(F)F)c1]

M. P.: 141° C.; IR (CHCl₃): ν 3383, 3208, 3020, 1662, 1635, 1541, 1456, 1352, 1280, 1215, 1147, 912, 757 cm⁻¹; ¹H NMR (CDCl₃, 500 MHz): δ 8.26 (s, 4H), 8.38 (s, 2H), 9.94 (s, 1H), 10.02 (s, 1H) ppm; ¹³C NMR (CDCl₃, 125 MHz): δ 125.6 (t), 127.7 (d), 128.3 (d), 132.2 (s), 132.5 (s), 166.5 (s), 182.2 (s) ppm; ESI-MS (m/z): 557.28 [M⁺1].

### 3.14 N-(4-chloro-2-hydroxy-5-nitrophenylcarbamothioyl)-3,5-bis(trifluoromethyl)benzamide (TU14)

The general procedure D was followed. ammoniumthiocyanate (55 mg, 0.72 mmol) 3,5-bis(trifluromethyl)benzoyl chloride (0.36 mmol) 2-amino-5-chloro-4-nitrophenol (68 mg, 0.36 mmol) were used to obtain 14 (166 mg, 94%) as yellow solid.

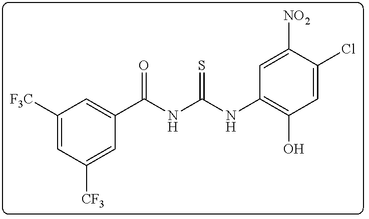 [SMILES: O=C(NC(=S)Nc1cc([N+](=O)[O-])c(Cl)cc1O)c1cc(C(F)(F)F)cc(C(F)(F)F)c1]

M. P.: 154° C.; IR (CHCl₃): ν 3525, 3257, 3020, 1669, 1592, 1521, 1408, 1331, 1280, 1216, 1190, 1148, 757 cm⁻¹; ¹H NMR (CDCl₃, 200 MHz): δ 7.64 (s, 1H), 8.18 (s, 1H), 8.24 (s, 1H), 8.40 (s, 2H), 8.79 (s, 1H), 9.48 (s, 1H), 12.93 (s, 1H) ppm; ¹³C NMR (CDCl₃+DMSO-d₆, 50 MHz): δ 110.9 (s), 115.8 (s), 122.4 (s), 125.5 (t, 2C), 129.0 (d, 2C), 130.5 (s), 130.9 (s), 131.2 (s), 133.5 (s), 142.4 (s), 146.7 (s), 165.0 (s), 177.3 (s) ppm; ESI-MS (m/z): 486.02 M⁺].

### 3.15 N-((4-methoxy-[1,1′-biphenyl]-3-yl)carbamothioyl)-3,5-bis(trifluoromethyl)benzamide (TU15)

The general procedure D was followed. Ammoniumthiocyanate (55 mg, 0.72 mmol) 3,5-bis(trifluromethyl)benzoyl chloride (0.36 mmol) 4-methoxy biphenyl-3-amine (72 mg, 0.36 mmol) were used to obtain 15 (164 mg, 91%) as yellow solid.

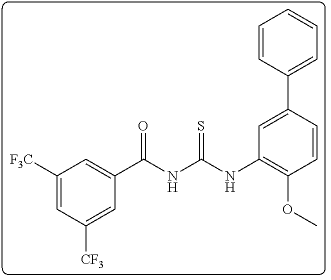 [SMILES: COc1ccc(-c2ccccc2)cc1NC(=S)NC(=O)c1cc(C(F)(F)F)cc(C(F)(F)F)c1]

M. P.: 142° C.; IR (CHCl₃): ν 3584, 3362, 3020, 1675, 1608, 1542, 1457, 1280, 1215, 1149, 1123, 754 cm⁻¹; ¹H NMR (CDCl₃, 200 MHz): δ 4.02 (s, 3H), 7.06 (d, J=8.6 Hz, 1H), 7.33-7.37 (m, 1H), 7.45-7.65 (m, 5H), 8.15 (s, 1H), 8.38 (s, 2H), 9.15 (d, J=2.2 Hz, 1H), 9.51 (s, 1H), 12.55 (s, 1H) ppm; ¹³C NMR (CDCl₃, 50 MHz): δ 56.3 (s), 110.9 (s), 114.2 (s), 121.3 (s), 125.3 (s), 126.7 (s), 126.9 (s), 127.0 (s), 127.8 (t), 0.7 (s), 128.8 (s), 133.3 (s), 133.5 (s), 134.1 (s), 140.2 (s), 149.9 (s), 163.5 (s), 175.6 (s) ppm; ESI-MS (m/z): 499.11 [M+1]⁺.

### 3.16 N-(2-methoxy-5-(trifluromethyl)phenylcarbamothioyl)-3,5 bis(trifluoromethyl)benzamide (TU16)

The general procedure D was followed ammoniumthiocyanate (55 mg, 0.72 mmol) 3,5-bis(trifluromethyl)benzoyl chloride (0.36 mmol) 2-methoxy-5-(trifluoromethyl)aniline (69 mg, 0.36 mmol) were used to obtain 16 (152 mg, 86%) as yellow solid.

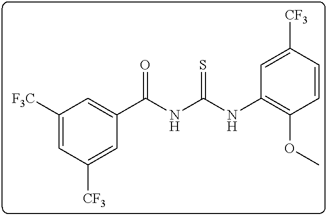 [SMILES: COc1ccc(C(F)(F)F)cc1NC(=S)NC(=O)c1cc(C(F)(F)F)cc(C(F)(F)F)c1]

M. P.: 164° C.; IR (CHCl₃): ν 3584, 3362, 3020, 1675, 1608, 1542, 1457, 1280, 1215, 1149, 1123, 754 cm⁻¹; ¹H NMR (CDCl₃, 200 MHz): δ 4.05 (s, 3H), 7.05 (d, J=8.7 Hz, 1H), 7.50 (dd, J=2.1, 8.7 Hz, 1H), 8.16 (s, 1H), 8.38 (s, 2H), 9.21 (d, J=2.1 Hz, 1H), 9.24 (s, 1H), 12.84 (s, 1H) ppm; ¹³C NMR (CDCl₃, 50 MHz): δ 56.4 (s), 110.3 (s), 119.6 (s), 124.1 (s), 126.9 (t, 2C), 127.8 (d, 2C), 128.1 (s, 2C), 130.0 (s), 130.8 (s), 134.9 (s), 148.7 (s), 165.1 (s), 174.1 (s) ppm; ESI-MS (m/z): 513.2 [M+Na]⁺.

### 3.17 N-(2-hydroxyphenylcarbamothioyl)-3,5-dimethoxybenzamide (TU17)

The general procedure D was followed ammoniumthiocyanate (76 mg, 1.0 mmol) 3,5-dimethoxybenzoyl chloride (0.5 mmol) 2-aminophenol (55 mg, 1.0 mmol) were used to obtain 17 (161 mg, 97%) as yellow solid.

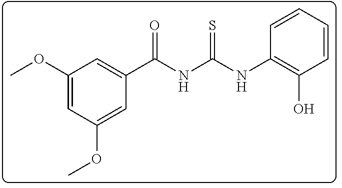 [SMILES: COc1cc(OC)cc(C(=O)NC(=S)Nc2ccccc2O)c1]

M. P.: 156° C.; IR (CHCl₃): ν 3422, 3020, 1651, 1589, 1553, 1523, 1461, 1361, 1218, 1205, 1138, 1051, 754 cm⁻¹; ¹H NMR (CDCl₃, 200 MHz): δ 3.86 (s, 6H), 6.71 (t, J=2.3 Hz, 1H), 6.97 (d, J=2.2 Hz, 1H), 7.04 (d, J=1.2, 8.0 Hz, 1H), 7.10 (dd, J=0.8, 8.0 Hz, 1H), 7.31 (dd, J=1.2, 8.0 Hz, 1H), 7.47 (dd, J=1.0, 8.0 Hz, 1H), 9.14 (s, 1H), 12.59 (s, 1H) ppm; ¹³C NMR (CDCl₃, 50 MHz): δ 55.7 (s, 2C), 105.4 (s, 2C), 105.8 (s), 119.7 (s), 121.7 (s), 125.0 (s), 125.8 (s), 128.9 (s), 133.3 (s), 149.5 (s), 161.3 (s, 2C), 167.2 (s), 167.2 (s), 177.4 (s) ppm; HRMS ESI calcd for [M+Na]⁺ C₁₆H₁₆N₂O₄SNa 355.0728. found 355.0723.

### 3.18 N-(4-chloro-2-hydroxyphenylcarbamothioyl)-3,5-dimethoxybenzamide (TU18)

The general procedure D was followed. ammoniumthiocyanate (76 mg, 1.0 mmol) 3,5-dimethoxybenzoyl chloride (0.5 mmol) 2-amino-5-chlorophenol (72 mg, 0.5 mmol) were used to obtain 18 (172 mg, 94%) as yellow solid.

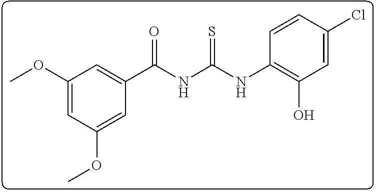 [SMILES: COc1cc(OC)cc(C(=O)NC(=S)Nc2ccc(Cl)cc2O)c1]

M. P.: 170° C.; IR (CHCl₃): ν 3391, 3020, 1657, 1592, 1555, 1534, 1456, 1370, 1218, 1207, 1159, 1066, 770 cm⁻¹; ¹H NMR (CDCl₃+DMSO-d₆, 200 MHz): δ 3.86 (s, 6H), 6.68 (t, J=2.3 Hz, 1H), 6.86 (dd, J=2.3, 8.7 Hz, 1H), 7.01 (d, J=2.3 Hz, 1H), 7.04 (d, J=2.3 Hz, 2H), 8.53 (d, J=8.7 Hz, 1H), 9.64 (s, 1H), 12.84 (s, 1H) ppm; ¹³C NMR (CDCl₃+DMS-d₆, 50 MHz): δ 55.4 (s, 2C), 105.4 (s, 2C), 106.9 (s), 115.5 (s), 118.6 (s), 123.7 (s), 124.7 (s), 131.0 (s), 133.7 (s), 149.6 (s), 160.7 (s, 2C), 166.6 (s), 176.7 (s) ppm; ESI-MS (m/z): 388.67 [M+Na]⁺; HRMS ESI calcd for (MH⁺) C₁₆H₁₆N₂O₄ClS 367.0519. found 367.0514, calcd for [M+Na]⁺ C₁₆H₁₅N₂O₄ClSNa 389.0333. found 389.0333.

### 3.19 N-(5-chloro-2-hydroxyphenylcarbamothioyl)-3,5-dimethoxybenzamide (TU19)

The general procedure D was followed. ammoniumthiocyanate (76 mg, 1.0 mmol) 3,5-dimethoxybenzoyl chloride (0.5 mmol) 2-amino-4-chlorophenol (72 mg, 0.5 mmol) were used to obtain 19 (175 mg, 95%) as yellow solid.

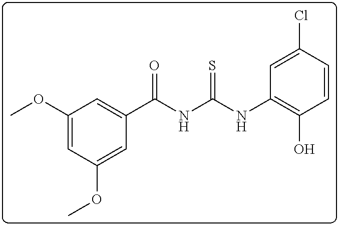 [SMILES: COc1cc(OC)cc(C(=O)NC(=S)Nc2cc(Cl)ccc2O)c1]

M. P.: 196° C.; IR (CHCl₃): ν 3407, 3020, 1661, 1598, 1561, 1526, 1496, 1371, 1215, 1159, 1065, 757 cm⁻¹; ¹H NMR (CDCl₃+DMSO-d₆, 200 MHz): δ 3.86 (s, 6H), 6.68 (t, J=2.3 Hz, 1H) 6.94 (d, J=8.7 Hz, 1H), 7.04 (d, J=2.2 Hz, 2H), 7.07 (d, J=2.3 Hz, 1H), 8.70 (d, J=2.3 Hz, 1H), 9.48 (s, 1H), 12.90 (s, 1H) ppm; ¹³C NMR (CDCl₃+DMS-d₆, 50 MHz): δ 55.4 (s, 2C), 105.3 (s), 105.5 (s, 2C), 115.9 (s), 122.1 (s), 125.7 (s), 126.9 (s), 133.6 (s), 147.3 (s), 160.6 (s, 2C), 166.7 (s), 176.7 (s) ppm; ESI-MS (m/z): 388.67 [M+Na]⁺; HRMS ESI calcd for (MH⁺) C₁₆H₁₆N₂O₄Cl S 367.0519. found 367.0514, calcd for [M+Na]⁺ C₁₆H₁₃N₂O₄ClSNa 389.0334. found 389.0333.

### 3.20 N-(2-hydroxy-4-methylphenylcarbamothioyl)-3,5-dimethoxybenzamide (TU20)

The general procedure D was followed. ammoniumthiocyanate (76 mg, 1.0 mmol) 3,5-dimethoxybenzoyl chloride (0.5 mmol) 2-amino-5-methylphenol (62 mg, 0.5 mmol) were used to obtain 20 (162 mg, 94%) as yellow solid.

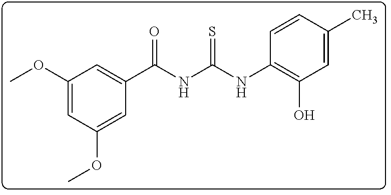

M. P.: 155° C.; IR (CHCl₃): ν 3422, 3020, 1670, 1603, 1542, 1524, 1498, 1371, 1216, 1161, 1065, 757 cm⁻¹; ¹H NMR (CDCl₃, 200 MHz): δ 2.34 (s, 3H), 3.85 (s, 6H), 6.70 (t, J=2.3 Hz, 1H), 6.83 (dd, J=1.3, 8.0 Hz, 1H), 6.91 (d, J=1.3 Hz, 1H), 6.97 (d, J=2.2 Hz, 2H), 7.30 (d, J=8.0 Hz, 1H), 9.14 (s, 1H), 12.49 (s, 1H) ppm; ¹³C NMR (CDCl₃, 50 MHz): δ 21.0 (s), 55.6 (s, 2C), 105.3 (s, 2C), 105.4 (s), 117.4 (s), 120.3 (s), 123.2 (s), 123.6 (s), 133.8 (s), 137.7 (s), 149.0 (s), 160.9 (s, 2C), 166.7 (s), 176.7 (s) ppm; ESI-MS (m/z): 369.08 [M+Na]⁺; HRMS ESI calcd for (MH⁺) C₁₇H₁₉N₂O₄S 347.1060. found 347.1060, calcd for [M+Na]⁺ C₁₇H₁₈N₂O₄SNa 369.0880. found 369.0879.

### 3.21 N-((4-hydroxy-[1,1′-biphenyl]-3-yl)carbamothioyl)-3,5-dimethoxybenzamide (TU21)

The general procedure D was followed. ammoniumthiocyanate (76 mg, 1.0 mmol) 3,5-dimethoxybenzoyl chloride (0.5 mmol) 3-aminobiphenyl-4-ol (93 mg, 0.5 mmol) were used to obtain 21 (183 mg, 92%) as yellow solid.

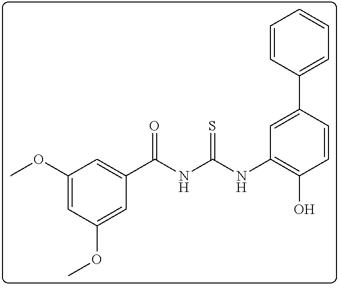 [SMILES: COc1cc(OC)cc(C(=O)NC(=S)Nc2cc(-c3ccccc3)ccc2O)c1]

M. P.: 171° C.; IR (CHCl₃): ν 3412, 3394, 3019, 2840, 1675, 1595, 1556, 1514, 1358, 1306, 1207, 1159, 1065, 758 cm⁻¹; ¹H NMR (CDCl₃, 200 MHz): δ 3.86 (s, 6H), 6.71 (t, J=2.3 Hz, 1H), 6.94 (d, J=2.2 Hz, 2H), 7.16 (d, J=8.3 Hz, 1H), 7.32-7.58 (m, 6H), 7.73 (d, J=2.1 Hz, 1H), 9.8 (s, 1H), 12.69 (s, 1H) ppm; ¹³C NMR (CDCl₃, 50 MHz): δ 55.7 (s, 2C), 105.4 (s, 2C), 105.8 (s), 120.0 (s), 123.5 (s), 126.0 (s), 126.8 (s), 127.1 (s), 127.5 (s), 128.8 (s, 2C), 133.3 (s), 134.8 (s), 148.9 (s), 161.3 (s, 2C), 167.3 (s), 177.4 (s) ppm; ESI-MS (m/z): 431.06 [M+Na]⁺; HRMS ESI calcd for (MH⁺) C₂₂H₂₁N₂O₄S 409.1216. found 409.1217, calcd for [M+Na]⁺ C₂₂H₂₀H₂O₄SNa 431.1036. found 431.1036.

### 3.22 N-(2-hydroxy-5-(trifluromethyl)phenylcarbamothioyl)-3,5-dimethoxybenzamide (TU22)

The general procedure D was followed. ammoniumthiocyanate (76 mg, 1.0 mmol) 3,5-dimethoxybenzoyl chloride (0.5 mmol) 2-amino-4-(trifluoromethyl)phenol (94 mg, 0.5 mmol) were used to obtain 22 (192 mg, 96%) as yellow solid.

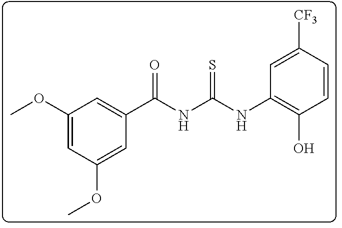 [SMILES: COc1cc(OC)cc(C(=O)NC(=S)Nc2cc(C(F)(F)F)ccc2O)c1]

M. P.: 173° C.; IR (CHCl₃): ν 3412, 3394, 3019, 2840, 1675, 1595, 1556, 1514, 1358, 1306, 1207, 1159, 1065, 758 cm⁻¹; ¹H NMR (CDCl₃, 200 MHz): δ 3.86 (s, 6H), 6.70 (t, J=2.3, 1H), 7.0 (d, J=2.2 Hz, 2H), 7.13 (d, J=8.3 Hz, 1H), 7.45 (dd, J=1.7, 8.3 Hz, 1H), 8.36 (d, J=1.9 Hz, 1H), 9.23 (s, 1H), 12.79 (s, 1H) ppm; ¹³C NMR (CDCl₃, 50 MHz): δ 55.5 (s, 2C), 105.2 (s), 105.4 (s, 2C), 115.4 (s), 120.0 (s), 120.1 (s), 123.5 (s), 123.6 (s), 126.2 (s), 133.7 (s), 151.6 (s), 160.9 (s, 2C), 166.6 (s), 176.9 (s) (s) ppm; HRMS ESI calcd for (MH⁺) C₁₇H₁₆N₂O₄F₃S 401.0777. found 401.0777, calcd for [M+Na]⁺ C₁₇H₁₅N₂O₄F₃SNa 423.0597. found 423.0597.

### 3.23 3,5-dimethoxy-N-(2-methoxy-5-(trifluoromethyl)phenylcarbamothioyl)benzamide (TU23)

The general procedure D was followed. ammoniumthiocyanate (76 mg, 1.0 mmol) 3,5-dimethoxybenzoyl chloride (0.5 mmol) 2-methoxy-5-(trifluoromethyl)aniline (96 mg, 0.5 mmol) were used to obtain 23 (193 mg, 94%) as yellow solid.

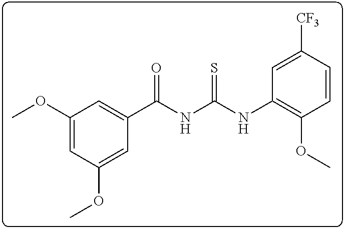

M. P.: 173° C.; IR (CHCl₃): ν 3412, 3394, 3019, 2840, 1675, 1595, 1556, 1514, 1358, 1306, 1207, 1159, 1065, 758 cm⁻¹; ¹H NMR (CDCl₃, 200 MHz): δ 3.86 (s, 6H), 4.02 (s, 3H), 6.69 (t, J=2.3 Hz 1H) 6.99 (d, J=2.3 Hz, 2H), 7.02 (d, J=2.3 Hz, 1H), 7.48 (dd, J=2.3, 8.6 Hz, 1H), 9.09 (s, 1H), 9.24 (d, J=2.0 Hz, 1H), 12.97 (s, 1H) ppm; HRMS ESI calcd for (MH⁺) C₁₈H₁₈H₂O₄F₃S 415.0939. found 415.0938.

### 3.24 N-(4-iodo-2-nitrophenylcarbamothioyl)-3,5-dimethylbenzamide (TU24)

The general procedure D was followed. Ammonium thiocyanate (90 mg, 1.2 mmol) 3,5-dimethylbenzoyl chloride (0.6 mmol) and 4-iodo-2-nitroaniline (158 mg, 0.6 mmol) were used to obtain 24 (207 mg, 84%) as yellow solid.

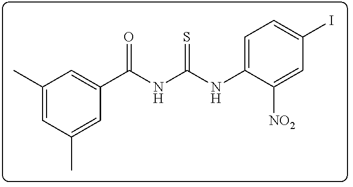 [SMILES: Cc1cc(C)cc(C(=O)NC(=S)Nc2ccc(I)cc2[N+](=O)[O-])c1]

M. P.: 117.6° C.; IR (CHCl₃): ν 3492, 3378, 2923, 1725, 1667, 1610, 1554, 1460, 1377, 1336, 1230, 1170 cm⁻¹; ¹H NMR (CDCl₃, 200 MHz): δ 2.42 (s, 6H), 7.25 (s, 2H), 7.56 (s, 2H), 7.99 (dd, J=2.1, 9.0 Hz, 1H), 8.58 (d, J=2.1 Hz, 1H), 8.80 (d, J=9.0 Hz, 1H), 11.21 (s, 1H) ppm; ¹³C NMR (CDCl₃, 50 MHz): δ21.3 (d, 2C), 84.5 (s), 123.6 (d), 125.1 (d, 2C), 133.6 (s), 134.0 (d), 134.5 (d), 135.1 (s), 136.5 (s), 138.8 (s, 2C), 144.6 (d), 165.9 (s), 177.8 (s) ppm; ESI-MS (m/z): 478.3 [M+Na]⁺; Anal. Calcd for C₁₆H₁₄IN₃O₃S: C, 42.21; H, 3.10; I, 27.87; N, 9.23 Found: C, 42.16; H, 3.07; I, 27.83; N, 9.19.

### 3.25 3,5-dimethyl-N-(3-nitrophenylcarbamothioyl)benzamide (TU25)

The general procedure D was followed. Ammonium thiocyanate (90 mg, 1.2 mmol) dimethylbenzoyl chloride (0.6 mmol) and 3-nitroaniline (144 mg, 0.6 mmol) were used to obtain 25 (146 mg, 84%) as yellow solid.

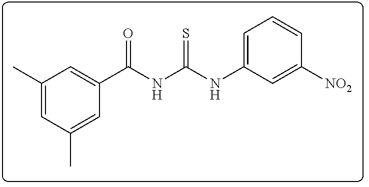 [SMILES: Cc1cc(C)cc(C(=O)NC(=S)Nc2cccc([N+](=O)[O-])c2)c1]

M. P.: 172.2° C.; IR (CHCl₃): ν 3351, 2922, 2858, 1558, 1538, 1466, 1416, 1215, 1077 cm⁻¹; ¹H NMR (CDCl₃, 200 MHz): δ 2.42 (s, 6H), 7.30 (s, 1H), 7.49 (s, 2H), 7.60 (t J=8.2, Hz, 1H), 7.07 (dd, J=1.3, 8.2 Hz, 1H), 8.15 (ddd, J=0.7, 2.0, 8.2 Hz, 1H), 8.77 (t, J=2.1, Hz, 1H), 9.15 (br s, 1H), 12.93 (br s, 1H) ppm; ¹³C NMR (CDCl₃, 50 MHz): δ21.2 (q), 118.9 (d), 121.3 (d), 125.3 (d), 129.6 (d), 129.7 (d), 131.1 (s), 135.7 (d), 138.7 (s), 139.2 (s), 148.3 (s), 167.6 (s), 178.9 (s) ppm; ESI-MS (m/z): 352.3 [M+Na]⁺; Anal. Calcd for C₁₆H₁₅N₃O₃S: C, 58.34; H, 4.59; N, 12.76. Found: C, 58.29; H, 4.54; N, 12.72.

### 3.26 Methyl 2-(3-(3,5-dimethylbenzoyl)thioureido)benzoate (TU26)

The general procedure D was followed. Ammonium thiocyanate (90 mg, 1.2 mmol) dimethylbenzoyl chloride (0.6 mmol) and methyl 2-aminobenzoate (158 mg, 0.6 mmol) were used to obtain 26 (156 mg, 87%) as yellow solid.

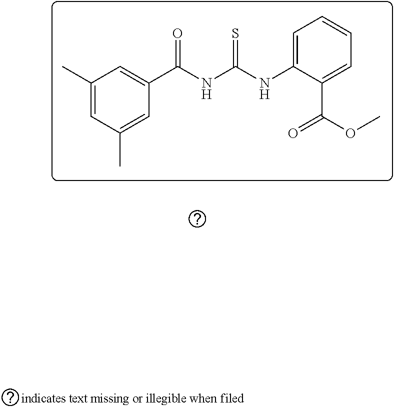

M. P.: 136.0° C.; IR (CHCl₃): ν 411, 3019, 2922, 2852, 1713, 1683, 1581, 1531, 1435, 1270, 1068 cm⁻¹; ¹H NMR (CDCl₃, 200 MHz): δ 2.40 (s, 6H), 3.96 (s, 3H), 7.31 (dt, J=1.1, 7.8 Hz, 1H), 7.54 (s, 2H), 7.60 (dt, J=1.6, 7.4 Hz, 1H), 8.04 (dd, J=1.6, 7.8 Hz, 1H), 8.51 (dd, J=0.6, 8.2 Hz, 1H), 9.12 (br s, 1H) ppm; ¹³C NMR (CDCl₃, 50 MHz): δ21.0 (q), 52.4 (q), 122.4 (s), 125.3 (d), 125.9 (d), 126.3 (d), 130.7 (d), 131.4 (s), 132.2 (d), 135.1 (d), 138.4 (s), 138.7 (s), 166.0 (s), 166.2 (s), 178.6 (s) ppm; ESI-MS (m/z): 365:5 [M+Na]⁺; Anal. Calcd for C₁₈H₁₈N₂O₃S: C, 63.14; H, 5.30; N, 8.18. Found: C, 63.09; H, 5.27; N, 8.14.

### 3.27 N-(2-hydroxynaphthalen-1-ylcarbamothioyl)-3,5-dimethylbenzamide (TU27)

The general procedure D was followed. Ammonium thiocyanate (90 mg, 1.2 mmol) dimethylbenzoyl chloride (0.6 mmol) and 1-aminonaphthalen-2-ol (0.6 mg, 0.6 mmol) were used to obtain 27 (163 mg, 91%) as yellow solid.

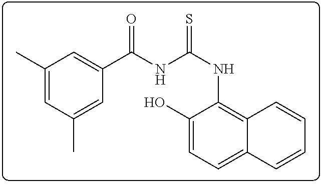 [SMILES: Cc1cc(C)cc(C(=O)NC(=S)Nc2c(O)ccc3ccccc23)c1]

M. P.: 214.7° C.; IR (CHCl₃): ν 3328, 3116, 2853, 2334, 1661, 1602, 1528, 1462, 1377, 1137, 746 cm⁻¹; ¹H NMR (CDCl₃, 200 MHz): δ 2.37 (s, 6H), 7.25 (d, J=8.9 Hz, 1H), 7.31 (s, 6H), 7.34 (d, J=7.5 Hz, 1H), 7.46 (ddd, J=1.0, 6.5, 8.1 Hz, 1H), 7.67 (d, J=8.5 Hz, 1H), 7.69 (s, 2H), 7.81 (d, J=8.9 Hz, 1H), 7.85 (d, J=8.1 Hz, 1H), 9.94 (s, 1H), 11.59 (s, 1H), 12.09 (s, 1H) ppm; ¹³C NMR (CDCl₃, 50 MHz): δ20.8 (q), 117.4 (s), 118.9 (d), 121.8 (d), 123.0 (d), 126.5 (d), 126.7 (d), 128.1 (d), 128.1 (d), 129.0 (d), 131.1 (s), 132.3 (s), 134.5 (d), 137.9 (s), 150.7 (s), 168.8 (s), 182.2 (s) ppm; ESI-MS (m/z): 373.4 [M+Na]⁺; Anal. Calcd for C₂₀H₁₈N₂O₂S: C, 68.55; H, 5.18; N, 7.99. Found: C, 68.50; H, 5.13; N, 7.94.

### 3.28 N-(3-chloro-6-hydroxy-2,4-dimethylphenylcarbamothioyl)-3,5-dimethylbenzamide (TU28)

The general procedure D was followed. Ammonium thiocyanate (90 mg, 1.2 mmol) dimethylbenzoyl chloride (0.6 mmol) and 2-amino-4-chloro-3,5-dimethylphenol (179 mg, 0.6 mmol) were used to obtain 28 (17.6 mg, 92%) as yellow solid.

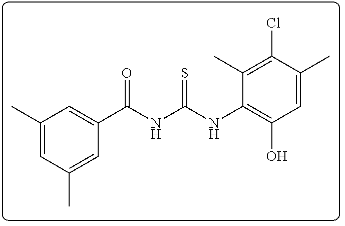

M. P.: 185.7° C.; IR (CHCl₃): ν 3322, 1681, 1528, 1460, 1377, 1296, 1164, 1051, 720 cm⁻¹; ¹H NMR (CDCl₃, 200 MHz): δ 2.37 (s, 3H), 2.39 (s, 3H), 2.42 (s, 6H), 6.23 (br s, 1H), 6.89 (s, 1H), 7.30 (s, 1H), 7.50 (s, 2H), 9.22 (s, 1H), 12.32 (s, 1H) ppm; ¹³C NMR (CDCl₃, 50 MHz): δ16.2 (q), 20.9 (q), 21.2 (q), 118.7 (d), 123.5 (s), 125.4 (d), 127.0 (s), 130.9 (s), 132.4 (s), 135.7 (d), 137.5 (s), 139.1 (s), 148.9 (s), 167.9 (s), 179.1 (s) ppm; ESI-MS (m/z): 385.8 [M+Na]⁺; Anal. Calcd for C₁₈H₁₉ClN₂O₂S: C, 59.58; H, 5.28; Cl, 9.77; N, 7.72. Found: C, 59.63; H, 5.32; Cl, 9.80; N, 7.70.

### 3.29 N-(4-fluoro-2-hydroxyphenylcarbamothioyl)-3,5-dimethylbenzamide (TU29)

The general procedure D was followed. Ammonium thiocyanate (90 mg, 1.2 mmol) dimethylbenzoyl chloride (0.6 mmol) and 2-amino-5-fluorophenol (133 mg, 0.6 mmol) were used to obtain 29 (149 mg, 89%) as yellow solid.

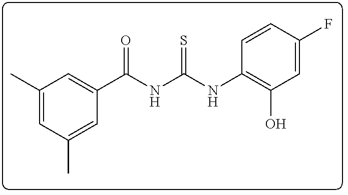 [SMILES: Cc1cc(C)cc(C(=O)NC(=S)Nc2ccc(F)cc2O)c1]

M. P.: 160.1° C.; IR (CHCl₃): ν 3228, 1615, 1598, 1521, 1466, 1376, 1255, 1017, 763 cm⁻¹; ¹H NMR (CDCl₃, 200 MHz): δ 2.42 (s, 6H), 6.74 (ddd, J=2.8, 7.8, 10.5 Hz, 1H), 6.84 (dd, J=2.8, 9.6 Hz, 1H), 7.29 (s, 1H), 7.32 (dd, J=6.0, 8.9 Hz, 1H), 7.48 (s, 2H), 9.17 (s, 1H), 12.56 (s, 1H) ppm; ¹³C NMR (CDCl₃, 50 MHz): δ 2.42 (s, 6H), 6.74 (ddd, J=2.8, 7.8, 10.5 Hz, 1H), 6.84 (dd, J=2.8, 9.6 Hz, 1H), 7.29 (s, 1H), 7.32 (dd, J=6.0, 8.9 Hz, 1H), 7.48 (s, 2H), 9.17 (s, 1H), 12.56 (s, 1H) ppm; ESI-MS (m/z): 341.3 [M+Na]⁺; Anal. Calcd for C₁₆H₁₅FN₂O₂S: C, 60.36; H, 4.75; F, 5.97; N, 8.80 Found: C, 60.30; H, 4.70; F, 5.92; N, 8.76.

### 3.30 N-(2-hydroxy-4-nitrophenylcarbamothioyl)-3,5-dimethylbenzamide (TU30)

The general procedure D was followed. Ammonium thiocyanate (90 mg, 1.2 mmol) dimethylbenzoyl chloride (0.6 mmol) and 2-amino-5-nitrophenol (161 mg, 0.6 mmol) were used to obtain 30 (162 mg, 89%) as yellow solid.

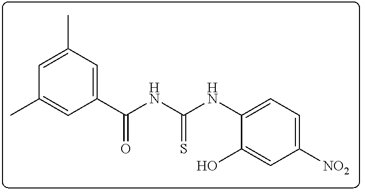 [SMILES: Cc1cc(C)cc(C(=O)NC(=S)Nc2ccc([N+](=O)[O-])cc2O)c1]

M. P.: 235.8° C.; IR (CHCl₃): ν 3368, 3323, 2503, 1595, 1564, 1521, 1462, 1376, 1337 cm⁻¹; ¹H NMR (CDCl₃, 200 MHz): δ 2.33 (s, 6H), 7.21 (s, 1H), 7.48 (s, 2H), 7.70 (s, 1H), 7.73 (d, J=8.8 Hz, 1H), 9.07 (d, J=8.8 Hz, 1H), 9.64 (s, 1H), 10.73 (s, 1H), 13.25 (s, 1H) ppm; ¹³C NMR (CDCl₃, 50 MHz): δ21.0 (q), 109.4 (d), 114.8 (d), 121.1 (d), 125.4 (d), 131.3 (s), 132.4 (s), 135.4 (d), 138.9 (s), 144.6 (s), 148.5 (s), 167.3 (s), 177.1 (s) ppm; ESI-MS (m/z): 368.4 [M+Na]⁺; Anal. Calcd for C₁₆H₁₅N₃O₄S: C, 55.64; H, 4.38; N, 12.17. Found: C, 55.59; H, 4.33; N, 12.12.

### 3.31 N-(2-hydroxy-5-nitrophenylcarbamothioyl)-3,5-dimethylbenzamide (TU31)

The general procedure D was followed. Ammonium thiocyanate (90 mg, 1.2 mmol) dimethylbenzoyl chloride (0.6 mmol) and 2-amino-4-nitrophenol (206 mg, 0.6 mmol) were used to obtain 31 (193 mg, 83%) as yellow solid.

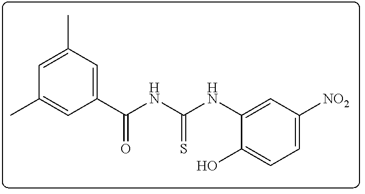 [SMILES: Cc1cc(C)cc(C(=O)NC(=S)Nc2cc([N+](=O)[O-])ccc2O)c1]

M. P.: 204.7° C.; IR (CHCl₃): ν 3368, 3291, 2506, 1637, 1598, 1560, 1520, 1459, 1377, 1350, 1105 cm⁻¹; ¹H NMR (CDCl₃, 200 MHz): δ 2.43 (s, 6H), 7.03 (d, j=9.0 Hz, 1H), 7.30 (s, 1H), 7.50 (s, 1H), 7.59 (s, 2H), 8.04 (dd, J=2.6, 9.0 Hz, 1H), 9.87 (d, J=2.6 Hz, 1H) ppm; ¹³C NMR (CDCl₃, 50 MHz): δ20.8 (q), 114.0 (d), 118.2 (d), 122.3 (d), 125.3 (d), 126.1 (s), 131.3 (s), 135.1 (d), 138.7 (s), 139.3 (s), 154.6 (s), 167.5 (s), 177.2 (s) ppm; ESI-MS (m/z): 368.3 [M+Na]⁺; Anal. Calcd for: C₁₆H₁₅N₃O₄S: C, 55.64; H, 4.38; N, 12.17. Found: C, 55.67; H, 4.43; N, 12.22.

### 3.32 N-(2-hydroxy-6-nitrophenylcarbamothioyl)-3,5-dimethylbenzamide (TU32)

The general procedure D was followed. Ammonium thiocyanat (90 mg, 1.2 mmol) dimethylbenzoyl chloride (0.6 mmol) and 2-amino-3-nitrophenol (161 mg, 0.6 mmol) were used to obtain 32 (162 mg, 89%) as yellow solid.

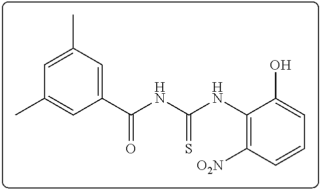 [SMILES: Cc1cc(C)cc(C(=O)NC(=S)Nc2c(O)cccc2[N+](=O)[O-])c1]

M. P.: 233.5° C.; IR ((CHCl₃): ν: 3483, 3384, 3286, 1675, 1521, 1463, 1376, 1160, 1107 cm⁻¹; ¹H NMR (CDCl₃, 200 MHz): δ 2.36 (s, 6H), 6.49 (dd, J=7.6, 8.8 Hz, 1H), 6.92 (dd, J=1.3, 7.6 Hz, 1H), 7.32 (s, 1H), 7.42 (d, J=7.6, 8.0 Hz, 1H), 7.65 (s, 2H), 10.74 (s, 1H), 11.73 (s, 1H), 12.25 (s, 1H) ppm; ¹³C NMR (CDCl₃, 50 MHz): δ20.9 (q), 21.0 (q), 114.7 (d), 115.5 (d), 117.2 (d), 126.5 (d), 126.66 (d), 137.4 (s), 138.0 (s), 146.5 (s), 147.3 (s), 154.2 (s), 168.9 (s), 181.4 (s) ppm; ESI-MS (m/z): 368.2 [M+Na]⁺; Anal. Calcd for C₁₆H₁₅N₃O₄S: C, 55.64; H, 4.38; N, 12.17 Found: C, 55.60; H, 4.33; N, 12.13.

### 3.33 N-(2-hydroxy-4-methylphenylcarbamothioyl)-3,5-dimethylbenzamide (TU33)

The general procedure D was followed. Ammonium thiocyanate (90 mg, 1.2 mmol) dimethylbenzoyl chloride (0.6 mmol) and 2-amino-5-methylphenol (128 mg, 0.6 mmol) were used to obtain 33 (151 mg, 91%) as yellow solid.

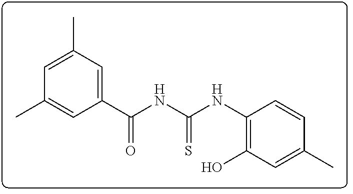

M. P.: 199.9° C.; IR (CHCl₃): ν 3472, 3402, 1655, 1596, 1551, 1459, 1360, 1213, 1107 cm⁻¹; ¹H NMR (CDCl₃, 200 MHz): δ 2.31 (s, 3H), 2.42 (s, 6H), 6.73 (d, J=8.1 Hz, 1H), 6.78 (s, 1H), 7.28 (s, 1H), 7.57 (s, 2H), 8.26 (d, J=8.1 Hz, 1H) ppm; ¹³C NMR (CDCl₃, 50 MHz): δ20.9 (q), 29.5 (q), 116.9 (d), 120.3 (d), 123.0 (s), 123.7 (d), 125.3 (d), 131.5 (s), 135.1 (d), 137.8 (s), 138.7 (s), 148.8 (s), 167.6 (s), 176.9 (s) ppm; ESI-MS (m/z): 337.6 [M+Na]⁺; Anal. Calcd for C₁₇H₁₈N₂O₂S: C, 64.94; H, 5.77; N, 8.91 Found: C, 64.89; H, 5.72; N, 8.88.

### 3.34 N-(2-hydroxy-5-methylphenylcarbamothioyl)-3,5-dimethylbenzamide (TU34)

The general procedure D was followed. Ammonium thiocyanate (90 mg, 1.2 mmol) dimethylbenzoyl chloride (0.6 mmol) and 2-amino-4-methylphenol (128 mg, 0.6 mmol) were used to obtain 34 (147 mg, 89%) as yellow solid.

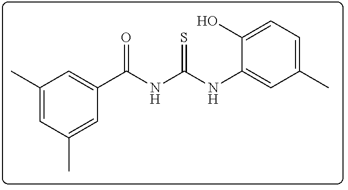

M. P.: 219.1° C.; IR (CHCl₃): ν 3409, 1659, 1599, 1565, 1460, 1375, 1218, 1101 cm⁻¹; ¹H NMR (CDCl₃, 200 MHz): δ 2.29 (s, 3H), 2.39 (s, 6H), 6.86 (s, 2H), 7.24 (s, 1H), 7.60 (s, 2H), 8.38 (d, J=11.0 Hz, 1H), 9.59 (s, 1H), 10.46 (s, 1H), 12.92 (s, 1H) ppm; ¹³C NMR (CDCl₃, 50 MHz): δ19.6 (q), 19.9 (q), 114.1 (d), 122.5 (d), 124.7 (s), 124.8 (d), 125.8 (d), 126.5 (s), 130.8 (s), 133.6 (d), 137.0 (s), 145.6 (s), 166.7 (s), 176.1 (s) ppm; ESI-MS (m/z): 337.2 [M+Na]⁺; Anal. Calcd for C₁₇H₁₈N₂O₂S: C, 64.94; H, 5.77; N; 8.91 Found: C, 65.00; H, 5.80; N, 8.93.

### 3.35 N-(5-chloro-2-hydroxyphenylcarbamothioyl)-3,5-dimethylbenzamide (TU35)

The general procedure D was followed. Ammonium thiocyanate (90 mg, 1.2 mmol) dimethylbenzoyl chloride (0.6 mmol) and 2-amino-4-chlorophenol (150 mg, 0.6 mmol) were used to obtain 35 (154 mg, 87%) as yellow solid.

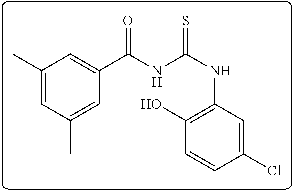 [SMILES: Cc1cc(C)cc(C(=O)NC(=S)Nc2cc(Cl)ccc2O)c1]

M. P.: 220.1° C.; IR (CHCl₃): ν 3442, 3248, 1657, 1601, 1533, 1377, 1152 cm⁻¹; ¹H NMR (CDCl₃, 200 MHz): δ 2.42 (s, 6H), 6.88 (d, J=8.6 Hz, 1H), 7.05 (dd, J=2.6, 8.7 Hz, 1H), 7.29 (s, 2H), 7.57 (s, 3H), 7.63 (s, 1H), 8.78 (d, J=2.5 Hz, 1H) ppm; ¹³C NMR (CDCl₃, 50 MHz): δ19.7 (q), 114.6 (d), 120.8 (d), 121.0 (s), 124.2 (d), 124.8 (d), 126.0 (s), 130.5 (s), 133.3 (d), 136.6 (s), 146.2 (s), 166.8 (s), 176.1 (s) ppm; ESI-MS (m/z): 357.7 [M+Na]⁺; Anal. Calcd for C₁₆H₁₅ClN₂O₂S: C, 57.40; H, 4.52; Cl, 10.59; N, 8.37 Found: C, 57.35; H, 4.47; Cl, 10.51; N, 8.32.

### 3.36 N-(5-chloro-2-hydroxy-4-nitrophenylcarbamothioyl)-3,5-dimethylbenzamide (TU36)

The general procedure D was followed. Ammonium thiocyanate (90 mg, 1.2 mmol) dimethylbenzoyl chloride (0.6 mmol) and 2-amino-4-chloro-5-nitrophenol (197 mg, 0.6 mmol) were used to obtain 36 (172 mg, 86%) as yellow solid.

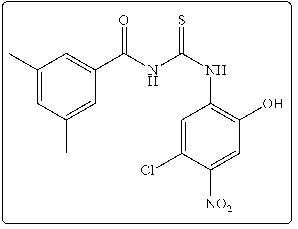 [SMILES: Cc1cc(C)cc(C(=O)NC(=S)Nc2cc(Cl)c([N+](=O)[O-])cc2O)c1]

M. P.: 249.7° C.; IR (CHCl₃) ν: 3342, 1653, 1590, 1558, 1377, 1325, 1105 cm⁻¹; ¹H NMR (CDCl₃, 200 MHz): δ 2.40 (s, 6H), 7.26 (s, 1H), 7.60 (s, 2H), 7.63 (s, 1H), 9.40 (s, 1H), 10.47 (br s, 1H) ppm; ¹³C NMR (CDCl₃, 50 MHz): δ20.6 (q), 111.0 (d), 116.1 (s), 122.4 (d), 125.3 (d), 130.9 (s), 131.1 (s), 134.6 (d), 138.0 (s), 142.4 (s), 146.7 (s), 167.3 (s), 177.2 (s) ppm; ESI-MS (m/z): 402.6 [M+Na]⁺; Anal. Calcd for C₁₆H₁₄ClN₃O₄S: C, 50.60; H, 3.72; N, 11.06 Found: C, 50.55; H, 3.68; N, 11.00.

### 3.37 N-(4-chloro-2-hydroxyphenylcarbamothioyl)-3,5-dimethylbenzamide (TU37)

The general procedure D was followed. Ammonium thiocyanate (90 mg, 1.2 mmol) dimethylbenzoyl chloride (0.6 mmol) and 2-amino-5-chlorophenol (150 mg, 0.6 mmol) were used to obtain 37 (163 mg, 93%) as yellow solid.

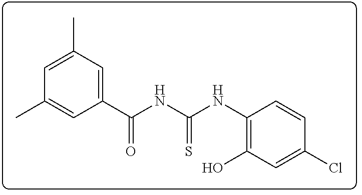 [SMILES: Cc1cc(C)cc(C(=O)NC(=S)Nc2ccc(Cl)cc2O)c1]

M. P.: 210.1° C.; IR (CHCl₃): ν: 3412, 3246, 1655, 1601, 1536, 11460, 1105 cm⁻¹; ¹H NMR (CDCl₃, 200 MHz): δ 2.39 (s, 6H), 6.81 (dd, J=2.3, 8.7 Hz, 1H), 6.98 (d, J=2.3 Hz, 1H), 7.24 (s, 1H), 7.60 (s, 2H), 8.63 (d, J=8.7 Hz, 1H), 10.53 (s, 1H), 10.68 (s, 1H) ppm; ¹³C NMR (CDCl₃, 50 MHz): δ19.5 (q), 113.5 (d), 116.6 (d), 122.4 (d), 123.6 (s), 124.7 (d), 128.7 (s), 130.4 (s), 133.0 (d), 136.3 (s), 148.4 (s), 166.7 (s), 176.0 (s) ppm; ESI-MS (m/z): 357.5 [M+Na]⁺; Anal. Calcd for C₁₆H₁₅ClN₂O₂S: C, 57.40; H, 4.52; N, 8.37. Found: C, 57.46; H, 4.57; N, 8.32.

### 3.38 N-(2-hydroxy-5-(trifluoromethyl)phenylcarbamothioyl)-3,5-dimethylbenzamide (TU38)

The general procedure D was followed. Ammonium thiocyanate (90 mg, 1.2 mmol) dimethylbenzoyl chloride (0.6 mmol) and 2-amino-4-(trifluoromethyl)phenol (185 mg, 0.6 mmol) were used to obtain 38 (168 mg, 87%) as yellow solid.

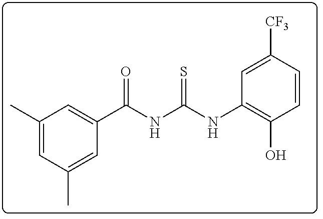 [SMILES: Cc1cc(C)cc(C(=O)NC(=S)Nc2cc(C(F)(F)F)ccc2O)c1]

M. P.: 208.6° C.; IR (CHCl₃): ν 3425, 3392, 1740, 1652, 1567, 1374, 1233, 1144 cm⁻¹; ¹H NMR (CDCl₃, 200 MHz): δ 2.40 (s, 6H), 7.08 (d, J=8.34 Hz, 1H), 7.26 (s, 1H), 7.30 (dd, J=2.27, 8.59 Hz, 1H), 7.61 (s, 2H), 9.15 (d, J=1.76, Hz, 1H), 10.47 (s, 6H) ppm; ¹³C NMR (CDCl₃, 50 MHz): δ20.2 (q, 2C), 114.2 (d), 118.9 (d, J=4.8 Hz), 119.5 (s), 122.2 (d, J=3.7 Hz), 125.0 (d, 2C), 125.6 (s), 126.2 (s), 130.9 (s), 133.9 (d), 137.3 (s, 2C), 150.8 (s, J=1.1 Hz), 167.0 (s), 176.7 (s) ppm; ESI-MS (m/z): 391.4 [M+Na]⁺; Anal. Calcd for C₁₇H₁₅F₃N₂O₂S: C, 55.43; H, 4.10; N, 7.60 Found: C, 55.38; H, 4.07; N, 7.56.

### 3.39 N-(3-chloro-6-methoxy-2,4-dimethylphenylcarbamathioyl)-3,5-dimethylbenzamide (TU39)

The general procedure D was followed. Ammonium thiocyanate (90 mg, 1.2 mmol) dimethylbenzoyl chloride (0.6 mmol) and 3-chloro-6-methoxy-2,4-dimethylaniline (194 mg, 0.6 mmol) were used to obtain 39 (179 mg, 90%) as yellow solid.

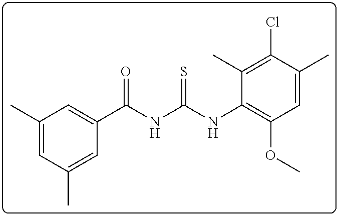

M. P.: 189.5-190.7° C.; IR (CHCl₃): ν 3434, 3155, 1672, 1602, 1585, 1519, 1460, 1377, 1327, 1148 cm⁻¹; ¹H NMR (CDCl₃, 200 MHz): δ 2.35 (s, 3H), 2.40 (s, 6H), 2.41 (s, 3H), 3.82 (s, 3H), 6.74 (s, 1H), 7.27 (s, 1H), 7.50 (s, 2H), 9.21 (s, 1H), 11.72 (s, 1H) ppm; ¹³C NMR (CDCl₃, 50 MHz): δ16.2 (q), 21.1 (q), 21.3 (q), 55.9 (q), 111.2 (d), 124.1 (s), 125.3 (d), 126.3 (s), 131.5 (s), 135.1 (s), 135.2 (d), 136.8 (s), 138.9 (s), 152.5 (s), 167.3 (s), 181.0 (s) ppm; ESI-MS (m/z): 399.5 [M+Na]⁺; Anal. Calcd for C₁₉H₂₁ClN₂O₂S: C, 60.55; H, 5.62; N, 7.43 Found: C, 60.50; H, 5.58; N, 7.38.

### 3.40 N-(2-methoxy-5-(trifluoromethyl)phenylcarbamothioyl)-3,5-dimethylbenzamide (TU40)

The general procedure D was followed. Ammonium thiocyanate (90 mg, 1.2 mmol) dimethylbenzoyl chloride (0.6 mmol) and 2-methoxy-5-(trifluoromethyl)aniline (199 mg, 0.6 mmol) were used to obtain 40 (173 mg, 86%) as yellow solid.

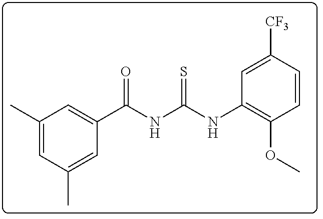

M. P.: 168.1° C.; IR (CHCl₃): ν 3395, 2853, 1677, 1601, 1562, 1519, 1462, 1377, 1313, 1115 cm⁻¹; ¹H NMR (CDCl₃, 200 MHz): δ 2.41 (s, 6H), 4.02 (s, 3H), 7.03 (d, J=8.8 Hz, 1H), 7.27 (s, 1H), 7.48 (d, J=9.6 Hz, 1H), 7.50 (s, 2H), 9.07 (s, 1H), 9.25 (d, J=1.4 Hz, 1H), 13.04 (s, 1H) ppm; ¹³C NMR (CDCl₃, 50 MHz): δ21.3 (d, 2C), 84.5 (s), 123.6 (d), 125.1 (d, 2C), 133.6 (s), 134.0 (d), 134.5 (d), 135.1 (s), 136.5 (s), 138.8 (s, 2C), 144.6 (d), 165.9 (s), 177.8 (s) ppm; ESI-MS (m/z): 405.4 [M+Na]⁺; Anal. Calcd for C₁₈H₁₇F₃N₂O₂S: C, 56.54; H, 4.48; N, 7.33 Found: C, 56.49; H, 4.43; N, 7.29.

### 3.41 N-(2-chloro-5-nitrophenylcarbamothioyl)-3,5-dimethylbenzamide (TU41)

The general procedure D was followed. Ammonium thiocyanate (90 mg, 1.2 mmol) dimethylbenzoyl chloride (0.6 mmol) and 2-chloro-5-nitroaniline (180 mg, 0.6 mmol) were used to obtain 41 (171 mg, 89%) as yellow solid.

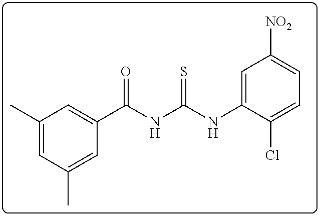 [SMILES: Cc1cc(C)cc(C(=O)NC(=S)Nc2cc([N+](=O)[O-])ccc2Cl)c1]

M. P.: 194-196° C.; IR (CHCl₃): ν 3422, 2925, 1676, 1606, 1578, 1523, 1459, 1376, 1349, 1309, 1206, 1147 cm⁻¹; ¹H NMR (CDCl₃, 200 MHz): δ 2.42 (s, 6H), 7.30 (s, 1H), 7.52 (s, 2H), 7.65 (d, J=8.8 Hz, 1H), 8.07 (dd, J=2.7, 8.8 Hz, 1H), 9.23 (s, 1H), 9.67 (d, J=2.7 Hz, 1H), 13.17 (s, 1H) ppm; ¹³C NMR (CDCl₃, 50 MHz): δ21.2 (q), 120.2 (d), 121.4 (d), 125.4 (d), 130.1 (d), 131.0 (s), 133.7 (s), 135.8 (d), 136.2 (s), 139.3 (s), 146.3 (s), 167.4 (s), 178.8 (s) ppm; ESI-MS (m/z): 368.6 [M+Na]⁺; Anal. Calcd for C₁₆H₁₄ClN₃O₃S: C, 52.82; H, 3.88; N, 11.55 Found: C, 52.77; H, 3.83; N, 11.50.

### 3.42 N-(2-hydroxy-4,6-dimethylphenylcarbamothioyl)-3,5-dimethylbenzamide (TU42)

The general procedure D was followed. Ammonium thiocyanate (90 mg, 1.2 mmol) dimethylbenzoyl chloride (0.6 mmol) and 2-amino-3,5-dimethylphenol (143 mg, 0.6 mmol) were used to obtain 42 (152 mg, 88%) as yellow solid.

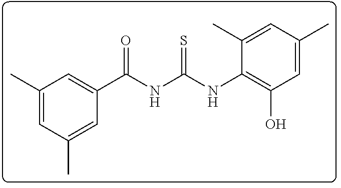

M. P.: 170.2° C.; IR (CHCl₃): ν 3376, 3138, 1798, 1739, 1665, 1590, 1519, 1463, 1377, 1340, 1208, 1146 cm⁻¹; ¹H NMR (CDCl₃, 200 MHz): δ 2.14 (s, 3H), 2.20 (s, 311), 2.35 (s, 6H), 6.54 (d, J=5.6 Hz, 2H), 7.29 (s, 1H), 7.63 (s, 2H), 9.37 (s, 1H), 11.41 (s, 1H), 11.78 (s, 1H) ppm; ¹³C NMR (CDCl₃, 50 MHz): δ17.9 (q), 20.7 (q), 20.8 (q), 114.2 (d), 121.3 (d), 121.9 (s), 126.3 (d), 132.0 (s), 134.4 (d), 136.1 (s), 137.4 (s), 137.7 (s), 152.2 (s), 168.5 (s), 181.2 (s) ppm; ESI-MS (m/z): 351.4 [M+Na]⁺; Anal. Calcd for C₁₈H₂₀N₂O₂S: C, 65.83; H, 6.14; N, 8.53 Found: C, 65.79; H, 6.09; N, 8.49.

### 3.43 N-(2-hydroxy-3,4,6-trimethylphenylcarbamothioyl)-3,5-dimethylbenzamide (TU43)

The general procedure D was followed. Ammonium thiocyanate (90 mg, 1.2 mmol) dimethylbenzoyl chloride (0.6 mmol) and 2-amino-4-chloro-3,5-dimethylphenol (158 mg, 0.6 mmol) were used to obtain 43 (157 mg, 87%) as yellow solid.

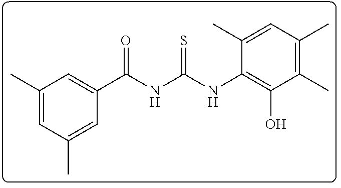

M. P.: 130.6-131.4° C.; IR (CHCl₃): ν 3318, 2728, 1673, 1604, 1519, 1462, 1377, 1166 cm⁻¹; ¹H NMR (CDCl₃, 200 MHz): δ 2.22 (s, 3H), 2.26 (s, 3H), 2.27 (s, 3H), 2.41 (s, 6H), 6.24 (br s, 1H), 6.72 (s, 1H), 7.29 (s, 1H), 7.50 (s, 2H), 9.19 (s, 1H), 12.25 (s, 1H) ppm; ¹³C NMR (CDCl₃, 50 MHz): δ12.0 (q), 17.9 (q), 19.9 (q), 21.1 (q), 122.6 (s), 124.1 (d), 124.4 (s), 125.3 (d), 130.1 (s), 131.1 (s), 135.5 (d), 137.7 (s), 139.0 (s), 148.5 (s), 167.8 (s), 178.6 (s) ppm; ESI-MS (m/z): 365.5 [M+Na]⁺; Anal. Calcd for C₁₉H₂₂N₂O₂S: C, 66.64; H, 6.48; N, 8.18 Found: C, 66.59; H, 6.43; N, 8.13.

### 3.44 N-(6-hydroxy-2,3,4-trimethylphenylcarbamothioyl)-3,5-dimethylbenzamide (TU44)

The general procedure D was followed. Ammonium thiocyanate (90 mg, 1.2 mmol) dimethylbenzoyl chloride (0.6 mmol) and 2-amino-3,4,5-trimethylphenol (158 mg, 0.6 mmol) were used to obtain 44 (147 mg, 82%) as yellow solid.

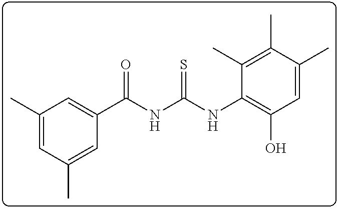

M. P.: 201.6-203° C.; IR (CHCl₃): ν 3339, 1667, 1596, 1532, 1463, 1377, 1156 cm⁻¹; ¹H NMR (CDCl₃, 200 MHz): δ 2.06 (s, 6H), 2.18 (s, 3H), 2.35 (s, 6H), 6.58 (s, 1H), 7.29 (s, 1H), 7.63 (s, 2H), 9.11 (s, 1H), 12.40 (s, 1H), 11.82 (s, 1H) ppm; ¹³C NMR (CDCl₃, 50 MHz): δ15.0 (q), 15.1 (q), 20.4 (q), 20.7 (q), 114.8 (d), 122.3 (s), 124.9 (s), 126.3 (d), 132.0 (s), 134.3 (s), 134.4 (d), 135.8 (s), 137.7 (s), 149.6 (s), 168.5 (s), 181.3 ppm; ESI-MS (m/z): 365.2 [M+Na]⁺; Anal. Calcd for C₁₉H₂₂N₂O₂S: C, 66.64; H, 6.48; N, 8.18 Found: C, 66.68; H, 6.52; N, 8.20.

### 3.45 N-(1-hydroxynaphthalen-2-ylcarbamothioyl)-3,5-dimethylbenzamide (TU45)

The general procedure D was followed. Ammonium thiocyanate (90 mg, 1.2 mmol) dimethylbenzoyl chloride (0.6 mmol) and 2-aminonaphthalen-1-ol (166 mg, 0.6 mmol) were used to obtain 45 (173 mg, 94%) as yellow solid.

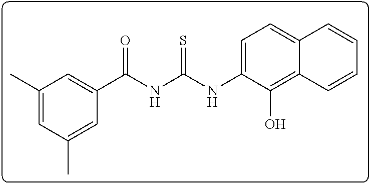 [SMILES: Cc1cc(C)cc(C(=O)NC(=S)Nc2ccc3ccccc3c2O)c1]

M. P.: 169.6° C.; IR (CHCl₃): ν 3317, 2726, 1741, 1666, 1601, 1527, 1463, 1377, 1214, 1137 cm⁻¹; ¹H NMR (CDCl₃, 200 MHz): δ 2.42 (s, 6H), 7.18 (br s, 1H), 7.29 (m, 1H), 7.49-7.56 (m, 51H), 7.79-7.84 (m, 1H), 8.41-8.45 (s, 2H), 9.24 (s, 1H), 12.79 (s, 1H) ppm; ¹³C NMR (CDCl₃, 50 MHz): δ21.1 (q, 2C), 119.8 (s), 121.4 (d), 123.0 (d), 123.2 (d), 125.3 (d, 2C), 125.8 (d), 126.8 (d), 127.3 (d), 127.4 (s), 131.0 (s), 133.7 (s), 135.6 (d), 139.1 (s), 145.4 (s), 168.0 (s), 177.9 (s) ppm; ESI-MS (m/z): 373.6 [M+Na]⁺; Anal. Calcd for C₂₀H₁₈N₂O₂S: C, 68.55; H, 5.18; N, 7.99 Found: C, 68.59; H, 5.23; N, 8.04.

### 3.46 N-(3-(3,5-dimethylbenzoyl)thioureido)-4-hydroxybenzoic acid (TU46)

The general procedure D was followed. Ammonium thiocyanate (90 mg, 1.2 mmol) dimethylbenzoyl chloride (0.6 mmol) and 3-amino-4-hydroxybenzoic acid (160 mg, 0.6 mmol) were used to obtain 46 (156 mg, 86%) as yellow solid.

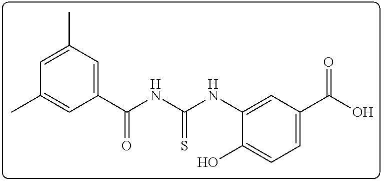 [SMILES: Cc1cc(C)cc(C(=O)NC(=S)Nc2cc(C(=O)O)ccc2O)c1]

M. P.: 265.0° C.; IR (CHCl₃): ν 3431, 3287, 2671, 1885, 1696, 1657, 1598, 1509, 1460, 1344, 1275, 1217, 1149 cm⁻¹; ¹H NMR (CDCl₃, 200 MHz): δ 2.36 (s, 6H), 7.03 (d, J=8.5 Hz, 1H), 7.30 (s, 1H), 7.64 (s, 2H), 7.61 (dd, J=2.0, 8.5 Hz, 1H), 9.25 (d, J=2.0 Hz, 1H), 11.15 (br s, 1H), 11.42 (s, 1H), 12.59 (br s, 1H), 13.06 (s, 1H) ppm; ¹³C NMR (CDCl₃, 50 MHz): δ20.7 (q), 114.8 (d), 120.9 (s), 124.6 (d), 125.9 (s), 126.3 (d), 128.3 (d), 131.8 (s), 134.5 (d), 137.8 (s), 153.0 (s), 167.0 (s), 168.4 (s), 177.9 (s) Ppm; ESI-MS (m/z): 373.4 [M+Na]⁺; Anal. Calcd for C₁₇H₁₆N₂O₄S: C, 59.29; H, 4.68; N, 8.13 Found: C, 59.34; H, 4.72; N, 8.17.

### 3.47 N-(2,6-dihydroxyphenylcarbamothioyl)-3,5-dimethylbenzamide (TU47)

The general procedure D was followed. Ammonium thiocyanate (90 mg, 1.2 mmol) dimethylbenzoyl chloride (0.6 mmol) and 2-aminobenzene-1,3-diol (130 mg, 0.6 mmol) were used to obtain 47 (148 mg, 89%) as yellow solid.

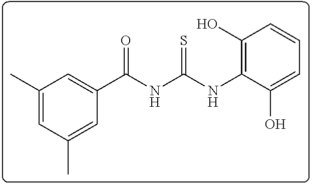 [SMILES: Cc1cc(C)cc(C(=O)NC(=S)Nc2c(O)cccc2O)c1]

M. P.: 207.2-209.1° C.; IR (CHCl₃): ν 3440, 3185, 2853, 1737, 1682, 1608, 1509, 1465, 1376, 1148, 1009 cm⁻¹; ¹H NMR (CDCl₃, 200 MHz): δ 2.39 (s, 6H), 6.49 (d, J=8.2 Hz, 2H), 7.99 (t, J=8.2 Hz, 1H), 7.24 (s, 1H), 7.61 (s, 2H), 8.87 (s, 2H), 10.86 (br s, 1H), 12.25 (s, 1H) ppm; ¹³C NMR (CDCl₃, 50 MHz): δ19.9 (q), 107.1 (d), 113.3 (s), 125.0 (d), 127.2 (d), 130.8 (s), 133.6 (d), 137.0 (s), 151.5 (s), 167.4 (s), 178.4 (s) ppm; ESI-MS (m/z): 339.2 [M+Na]⁺; Anal. Calcd for C₁₆H₁₆N₂O₃S: C, 60.74; H, 5.10; N, 8.85 Found: C, 60.69; H, 5.06; N, 8.81.

### 3.48 N-(2-hydroxy-4,6-bis(trifluoromethyl)phenylcarbamothioyl)-3,5-dimethylbenzamide (TU48)

The general procedure D was followed. Ammonium thiocyanate (90 mg, 1.2 mmol) dimethylbenzoyl chloride (0.6 mmol) and 2-amino-3,5-bis(trifluoromethyl)phenol (256 mg, 0.6 mmol) were used to obtain 48 (197 mg, 86%) as yellow solid.

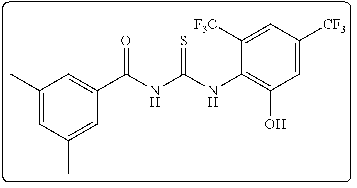 [SMILES: Cc1cc(C)cc(C(=O)NC(=S)Nc2c(O)cc(C(F)(F)F)cc2C(F)(F)F)c1]

M. P.: 167.5° C.; IR (CHCl₃) □ν: 3388, 2853, 1680, 1604, 1459, 1376, 1279, 1214, 1127, 761 cm⁻¹; ¹H NMR (CDCl₃, 200 MHz): δ 2.42 (s, 6H), 6.85 (s, 1H), 7.32 (s, 1H), 7.52 (s, 2H), 7.61 (s, 2H), 9.32 (br s, 1H), 12.84 (br s, 1H) ppm; ¹³C NMR (CDCl₃, 50 MHz): δ21.1 (q, 2C), 116.1 (d), 121.1 (s), 121.5 (d, J=3.7 Hz), 123.5 (s, J=32.3 Hz), 125.5 (d), 126.7 (s), 127.4 (s, J=30.1 Hz), 130.5 (s), 131.5 (s, J=34.5 Hz), 136.1 (d), 139.3 (s), 152.6 (s), 167.9 (s), 179.3 (s) ppm; ESI-MS (m/z): 459.1 [M+Na]⁺; Anal. Calcd for C₁₈H₁₄F₆N₂O₂S: C, 49.54; H, 3.23; N, 6.42 Found: C, 49.49; H, 3.19; N, 6.39.

### 3.49 N-(3-bromo-6-hydroxy-2,4-dimethylphenylcarbamothioyl)-3,5-dimethylbenzamide (TU49)

The general procedure D was followed. Ammonium thiocyanate (90 mg, 1.2 mmol) dimethylbenzoyl chloride (0.6 mmol) and 2-amino-4-bromo-3,5-dimethylphenol (225 mg, 0.6 mmol) were used to obtain 49 (184 mg, 92%) as yellow solid.

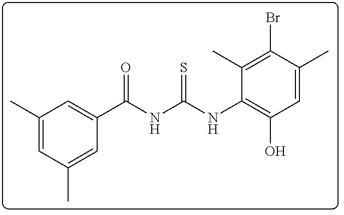

M. P.: 164.6-166.0° C.; IR (CHCl₃): ν 3325, 1679, 1605, 1515, 1403, 1211, 1157, 1024 cm⁻¹; ¹H NMR (CDCl₃, 200 MHz): δ 2.41 (s, 9H), 2.44 (s, 3H), 6.92 (s, 1H), 7.30 (s, 1H), 7.50 (s, 2H), 9.26 (s, 1H), 12.31 (s, 1H) ppm; ¹³C NMR (CDCl₃, 50 MHz): δ19.5 (q), 21.2 (q), 24.1 (q), 119.2 (d), 123.6 (s), 125.4 (d), 131.0 (s), 134.0 (s), 135.9 (d), 139.3 (s), 139.7 (s), 149.6 (s), 167.9 (s), 178.7 (s) ppm; ESI-MS (m/z): 429.2 [M+Na]⁺; Anal. Calcd for C₁₈H₁₉BrN₂O₂S: C, 53.08; H, 4.70; N, 6.88 Found: C, 53.11; H, 4.73; N, 6.93.

### 3.50 N-(4-hydroxybiphenyl-3-ylcarbamothioyl)-3,5-dimethylbenzamide (TU50)

The general procedure D was followed. Ammonium thiocyanate (90 mg, 1.2 mmol) dimethylbenzoyl chloride (0.6 mmol) and 3-aminobiphenyl-4-ol (193 mg, 0.6 mmol) were used to obtain 50 (184 mg, 93%) as yellow solid.

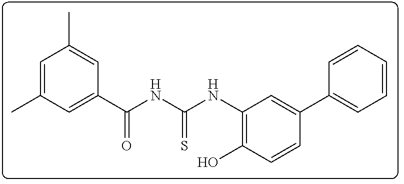 [SMILES: Cc1cc(C)cc(C(=O)NC(=S)Nc2cc(-c3ccccc3)ccc2O)c1]

M. P.: 117.6° C.; IR (CHCl₃): ν 3492, 3378, 2923, 1725, 1667, 1610, 1554, 1460, 1377, 1336, 1230, 1170 cm⁻¹; ¹H NMR (CDCl₃, 200 MHz): δ 2.42 (s, 6H), 7.17 (d, J=8.5 Hz, 1H), 7.30 (s, 1H); 7.36-7.40 (m, 1H), 7.43-7.48 (m, 2H), 7.50-7.60 (m, 5H), 7.69 (d, J=2.3 Hz, 1H), 9.22 (br s, 1H), 12.76 (br s, 1H) ppm; ¹³C NMR (CDCl₃, 50 MHz): δ21.3 (d, 2C), 84.5 (s), 123.6 (d), 125.1 (d, 2C), 133.6 (s), 134.0 (d), 134.5 (d), 135.1 (s), 136.5 (s), 138.8 (s, 2C), 144.6 (d), 165.9 (s), 177.8 (s) ppm; ESI-MS (m/z): 399.5 [M+Na]⁺; Anal. Calcd for C₂₂H₂₀N₂O₂S: C, 70.19; H, 5.35; N, 7.44 Found: C, 70.24; H, 5.38; N, 7.40.

### ADVANTAGES OF THE INVENTION

1. One pot reaction

2. The reaction is performed at 26° C.

1. A one pot room temperature process for preparation of acyl thioureas of formula (I) With yield greater than 80%, 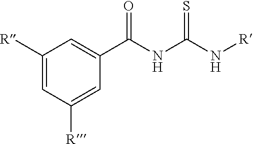
wherein, R′ is an aryl or a heteroarylene group substituted with one or more groups selected from hydrogen, alkyl, alkylene, alkynyl, alkoxy, alkenyloxy, halo, hydroxyl, nitro, amino, carboxyl, ester, halogenated hydrocarbon, aryl and heteroaryl, and R″ and R′″ are selected independently from hydrogen, alkyl, alkylene, alkynyl, alkoxy, alkenyloxy, halo, hydroxyl, nitro, amino and halogenated hydrocarbon,
the process comprising the steps of:
i. mixing aroyl chloride of formula (II) with ammonium isothiocyanate in the ratio ranging between 1:1 to 1:3 in acetonitrile followed by stirring for a period in the range of 3 to 5 minutes to obtain isothiocyanate; and 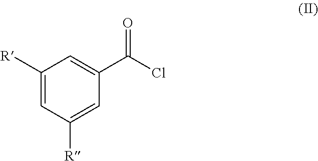
ii. adding the isothiocyanate as obtained in step (i) with a solution of an amine of general formula R′—NH2 in acetonitrile in the ratio ranging between 1:0.5 to 1:1.5 followed by stirring for a period in the range of 3 to 4 hr at room temperature in the range of 20 to 30° C. to obtain the acyl thioureas of formula (I).

2. The process of claim 1, wherein representative compounds of formula (I) include:
N-(2-hydroxyphenylcarbamothioyl)-3,5-bis(trifluoromethyl)benzamide
N-(2-hydroxy-5-nitrophenylcarbamothioyl)-3,5-bis(trifluoromethyl)benzamide
N-(2-hydroxy-4-nitrophenylcarbamothioyl)-3,5-bis(trifluoromethyl) benzamide
N-(4-chloro-2-hydroxyphenylcarbamothioyl)-3,5-bis(trifluoromethyl) benzamide
N-(5-chloro-2-hydroxyphenylcarbamothioyl)-3,5-bis(trifluoromethyl) benzamide
N-(2-hydroxy-5-methylphenylcarbamothioyl)-3,5-bis(trifluoromethyl)benzamide
N-(2-hydroxy-4-methylphenylcarbamothioyl)-3,5-bis(trifluoromethyl)benzamide
N-(2-hydroxy-3-nitrophenylcarbamothioyl)-3,5-bis(trifluoromethyl)benzamide
N-(2-hydroxy-5-(trifluromethyl)phenylcarbaniothioyl)-3,5-bis(trifluoromethyl)benzamide
N-(3-bromo-6-hydroxy-2,4-dimethylphenylcarbamothioyl)-3,5bis(trifluoromethyl) benzamide
N-(2-hydroxy-4,6-bis(trifluromethyl)phenylcarbamothioyl)-3,5 bis (trifluoromethyl) benzamide
N-((4-hydroxy-(1,1′-biphenyl]-3-yl)carbamothioyl)-3,5-bis(trifluoromethyl) benzamide
N,N′-thiocarbonylbis(3,5-bis(trifluoromethyl)benzamide)
N-(4-chloro-2-hydroxy-5-nitrophenylcarbamothioyl)-3,5-bis(trifluoromethyl)benzamide
N-((4-methoxy-(1,1′-biphenyl]-3-yl)carbamothioyl)-3,5-bis(trifluoromethyl) benzamide
N-(2-methoxy-5-(trifluromethyl)phenylcarbamothioyl)-3,5-bis (trifluoromethyl) benzamide
N-(2-hydroxyphenylcarbamothioyl)-3,5-dimethoxybenzamide
N-(4-chloro-2-hydroxyphenylcarbamothioyl)-3,5-dimethoxybenzamide
N-(5-chloro-2-hydroxyphenylcarbamothioyl)-3,5-dimethoxybenzamide
N-(2-hydroxy-4-methylphenylcarbamothioyl)-3,5-dimethoxybenzamide
N-((4-hydroxy-[1,1′-biphenyl]-3-yl)carbamothioyl)-3,5-dimethoxybenzamide
N-(2-hydroxy-5-(trifluromethyl)phenylcarbamothioyl)-3,5-dimethoxy benzamide 3,5-dimethoxy-N (2-methoxy-5-(trifluoromethyl)phenylcarbamothioyl)benzamide
N-(4-iodo-2-nitrophenylcarbamothioyl)-3,5-dimethylbenzamide
3,5-dimethyl-N-(3-nitrophenylcarbamothioyl)benzamide
Methyl 2-(3-(3,5-dimethylbenzoyl)thioureido)benzoate
N-(2-hydroxynaphthalen-1-ylcarbamothioyl)-3,5-dimethylbenzamide
N-(3-chloro-6-hydroxy-2,4-dimethylphenylcarbamothioyl)-3,5-dimethylbenzamide
N-(4-fluoro-2-hydroxyphenylcarbamothioyl)-3,5-dimethylbenzamide
N-(2 hydroxy-4-nitrophenylcarbamothioyl)-3,5-dimethylbenzamide
N-(2-hydroxy-5-nitrophenylcarbamothioyl)-3,5-dimethylbenzamide
N (2-hydroxy-4-methylphenylcarbamothioyl)-3,5-dimethylbenzamide
N-(2-hydroxy-6-nitrophenylcarbamothioyl)-3,5-dimethylbenzamide
N-(2-hydroxy-5-methylphenylcarbamothioyl)-3,5-dimethylbenzamide
N-(5-chloro-2-hydroxyphenylcarbamothioyl)-3,5-dimethylbenzamide
N-(5-chloro-2-hydroxy-4-nitrophenylcarbamothioyl)-3,5-dimethylbenzamide
N-(4-chloro-2-hydroxyphenylcarbamothioyl)-3,5-dimethylbenzamide
N-(3-chloro-6-methoxy-2,4-dimethylphenylcarbamothioyl)-3,5-dimethylbenzamide
N-(2-hydroxy-5-(trifluoromethyl)phenylcarbamothioyl)-3,5-dimethylbenzamide
N-(2-methoxy-5-(trifluoromethyl)phenylcarbamothioyl)-3,5-dimethylbenzamide
N-(2-chloro-5-nitrophenylcarbamothioyl)-3,5-dimethylbenzamide
N-(2-hydroxy-4,6-dimethylphenylcarbamothioyl)-3,5-dimethylbenzamide
N-(2-hydroxy-3,4,6-trimethylphenylcarbamothioyl)-3,5-dimethylbenzamide
N-(6-hydroxy-2,3,4-trimethylphenylcarbamothioyl)-3,5-dimethylbenzamide
N-(1-hydroxynaphthalen-2-ylcarbamothioyl)-3,5-dimethylbenzamide
3-(3-(3,5-dimethylbenzoyl)thioureido)-4-hydroxybenzoic acid
N-(2,6-dihydroxyphenylcarbamothioyl)-3,5-dimethylbenzamide
N-(2-hydroxy-4,6-bis(trifluoromethyl)phenylcarbamothioyl)-3,5-dimethylbenzamide
N-(3-bromo-6-hydroxy-2,4-dimethylphenylcarbamothioyl)-3,5-dimethylbenzamide
N-(4-hydroxybiphenyl-3-ylcarbamothioyl)-3,5-dimethylbenzamide.

3. The process of claim 1, wherein the yield of the acyl thioureas of formula (I) is in the range of 82% to 97%.

4. The process of claim 1, wherein the mixed ratio of the aroyl chloride of formula (II) and the ammonium isothiocyanate is 1:2.
